(19) United States
(12) Patent Application Publication
(10) Pub. No.: US 2025/0391044 A1
(43) Pub. Date: Dec. 25, 2025

(54) INFORMATION PROCESSING APPARATUS, RADIOGRAPHIC IMAGING SYSTEM, METHOD FOR INFORMATION PROCESSING, AND STORAGE MEDIUM

(71) Applicant: CANON KABUSHIKI KAISHA, Tokyo (JP)

(72) Inventors: TOMOHIRO KAWANISHI, Tokyo (JP); MANABU YAMAZOE, Tokyo (JP)

(21) Appl. No.: 19/242,577

(22) Filed: Jun. 18, 2025

(30) Foreign Application Priority Data
Jun. 21, 2024 (JP) 2024-100393
Nov. 27, 2024 (JP) 2024-206046

(51) Int. Cl. G06T 7/70; A61B 6/00; G06V 10/25; G06V 40/10

(52) U.S. Cl. CPC G06T 7/70; A61B 6/00; G06V 10/25; G06V 40/10

(57) ABSTRACT

An information processing apparatus according to an embodiment of the present disclosure includes at least one memory storing instructions; and at least one processor that, upon execution of the instructions, is configured to operate as: an acquisition unit configured to acquire imaging order information and an optical image related to radiographic imaging, an identification unit configured to identify a radiation exposure field using the imaging order information and the optical image, a detection unit configured to detect a human body part in the optical image, and an analysis unit configured to, when a plurality of human body parts is detected in the optical image, analyze a human body part closest to the radiation exposure field using the optical image.

### BACKGROUND

### Field

The present disclosure relates to an information processing apparatus, a radiographic imaging system, a method for information processing, and a storage medium.

### Description of the Related Art

In recent radiographic imaging for medical examinations, imaging support using optical images has become common, where the imaging part is captured with an optical camera, and additional information derived by analyzing the optical image is provided to the operator together with a live image. For example, Japanese Patent Laid-Open No. 2020-199163 proposes a technique for efficient radiographic imaging independent of the skill and experience of the operator by determining the shooting posture of the object from an optical image and outputting information on the suitability of the shooting posture. Japanese Patent Laid-Open No. 2022-110441 proposes a technique for stably displaying a detection frame indicating the region of the object detected from a live image.

In the imaging part, if the object is an aged person or a child, the radiology technician may adjust the shooting position of the object while supporting the object. In this case, a person (in this case, the radiology technician) other than the object can appear in the optical image. Depending on the lighting environment of the imaging room, the contrast between the background and the object can be decreased. If multiple persons appear in the optical image, a person other than the object can be recognized as the object. If the contrast between the background and the object decreases, the recognition of the object itself may become unstable. This may make it impossible to appropriately determine the suitability of the imaging posture of the object.

### SUMMARY OF THE INVENTION

The present disclosure provides an information processing apparatus configured to appropriately determine the suitability of the imaging posture of the object even when multiple objects appear in the optical image.

An information processing apparatus according to an embodiment of the present disclosure includes at least one memory storing instructions; and at least one processor that, upon execution of the instructions, is configured to operate as: an acquisition unit configured to acquire imaging order information and an optical image related to radiographic imaging, an identification unit configured to identify a radiation exposure field using the imaging order information and the optical image, a detection unit configured to detect a human body part in the optical image, and an analysis unit configured to, when a plurality of human body parts is detected in the optical image, analyze a human body part closest to the radiation exposure field using the optical image.

Further features of the present disclosure will become apparent from the following description of exemplary embodiments with reference to the attached drawings.

### BRIEF DESCRIPTION OF THE DRAWINGS

FIG. 1 is a diagram illustrating, in outline, the configuration of a radiographic imaging system according to an embodiment of the present disclosure.

FIG. 2 is a diagram illustrating, in outline, the configuration of an information processing apparatus according to an embodiment of the present disclosure.

FIG. 3 is a flowchart showing processing steps according to Related Example 1.

FIG. 4 is a diagram illustrating an example of an optical image according to Related Example 1.

FIG. 5 is a diagram illustrating an example of the output of a skeleton estimation unit according to Related Example 1.

FIG. 6 is a flowchart showing an object information determination process according to Related Example 1.

FIG. 7 is a diagram illustrating the arrangement of a radiation generation unit in a camera coordinate system.

FIG. 8 is a flowchart of an example of a process using object information according to Related Example 1.

FIG. 9 is a flowchart showing processing steps according to Related Example 2.

FIG. 10 is a diagram illustrating an example of an optical image according to Related Example 2.

FIG. 11 is a diagram illustrating an example of the output of a skeleton estimation unit according to Related Example 2.

FIG. 12 is a flowchart showing an example of an object information determination process according to Related Example 2.

FIG. 13 is a diagram illustrating another example of an optical image according to Related Example 2.

FIG. 14 is a flowchart showing another example of the object information determination process according to Related Example 2.

FIG. 15 is a flowchart showing processing steps according to Related Example 3.

FIG. 16A is a flowchart showing processing steps according to a first embodiment.

FIG. 16B is a flowchart showing processing steps according to the first embodiment.

FIG. 17 is a diagram illustrating an example of an optical image according to the first embodiment.

FIG. 18 is a diagram illustrating an example of the output of an object-information determination unit according to the first embodiment.

FIG. 19 is a flowchart showing processing steps according to a second embodiment.

FIG. 20 is a diagram illustrating an example of the output of an object-information determination unit according to the second embodiment.

FIG. 21 is a flowchart showing processing steps according to a third embodiment.

FIG. 22 is a diagram illustrating an example of an optical image according to the third embodiment.

FIG. 23 is a diagram illustrating an example of the output of an object-information determination unit according to the third embodiment.

FIG. 24 is a flowchart showing processing steps according to a fourth embodiment.

### DESCRIPTION OF THE EMBODIMENTS

Exemplary embodiments and examples for implementing the present disclosure will be described hereinbelow with reference to the drawings. It is to be understood that the sizes, materials, shapes, and relative positions of the components described in the following embodiments and examples may be freely changed according to the configuration of the apparatus to which the present disclosure is applied or various conditions. The same reference sign is used to indicate the same or similar component throughout the drawings.

The term radiation may include electromagnetic radiation, such as X-rays and gamma rays, and corpuscular radiation, such as alpha rays, beta rays, particle beams, proton beams, heavy ion beams, and meson beams.

A machine learning model refers to a learning model created using machine learning algorithms. Specific algorithms for machine learning include the nearest neighbor method, the naive Bayes method, decision trees, and support vector machines. Another example is deep learning, which autonomously generates features for learning and connection weighting coefficients using neural networks. Example of the algorithms using decision trees are LightGBM and XGBoost, which utilize gradient boosting. An appropriate one of the algorithms may be applied to the following embodiments. Training data, also referred to as labeled data, consists of pairs of input data and corresponding output data. The output data of training data is also referred to as correct answer data.

A trained model refers to a model trained in advance using appropriate training data (labeled data) in accordance with any machine learning algorithm, such as deep learning. However, the trained model, which is obtained in advance using appropriate training data, is not limited to no further learning, it can also undergo additional training. Additional training can be performed even after the apparatus is installed at the user's location.

### EMBODIMENTS

### Schematic Configuration

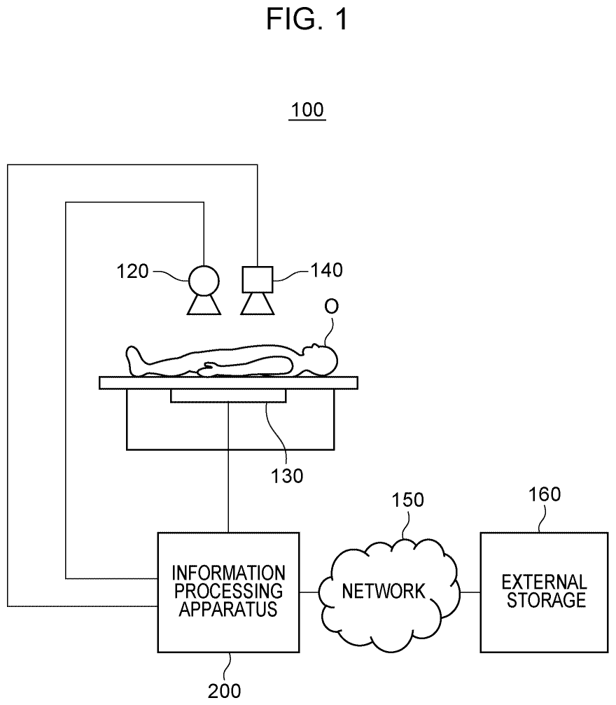

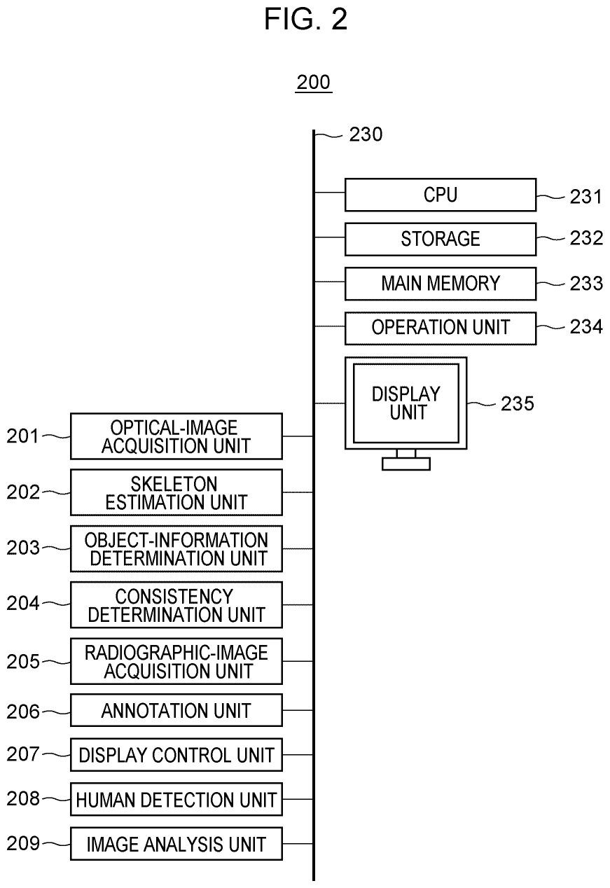

First, a radiographic imaging system, an information processing apparatus, and a method for information processing according to an embodiment of the present disclosure will be described with reference to FIGS. 1 and 2. An embodiment of the present disclosure is applied to a radiographic imaging system 100 and an information processing apparatus 200, as shown in FIGS. 1 and 2. FIG. 1 illustrates, in outline, the configuration of the radiographic imaging system 100 according to an embodiment of the present disclosure. FIG. 2 illustrates, in outline, the configuration of the information processing apparatus 200 according to an embodiment of the present disclosure. Although FIG. 1 illustrates a state in which an object O is in a supine position, the object O may also be in a standing position or seated position, for example. The imaging table used to support the object O may also be a table adapted to the posture of the object O.

The radiographic imaging system 100 includes the information processing apparatus 200, a radiation generation unit 120, a radiation detector 130, and a camera 140. The information processing apparatus 200 connects to the radiation generation unit 120, the radiation detector 130, and the camera 140 and can control them. The information processing apparatus 200 can process and analyze various images acquired using the radiation detector 130 and the camera 140. The information processing apparatus 200 also connects to an external storage 160, such as a server, via any network 150, such as the Internet or an intranet, to send and receive data to/from the external storage 160. The external storage 160 may directly connect to the information processing apparatus 200.

The radiation generation unit 120 includes a radiation generator, such as an X-ray tube, a collimator, a collimator lamp, etc., and can emit radiation beams under the control of the information processing apparatus 200. The radiation beams emitted from the radiation generation unit 120 pass through the object O while attenuating and enter the radiation detector 130.

The radiation detector 130 can detect the incoming radiation beams and send a signal corresponding to the detected radiation beams to the information processing apparatus 200. The radiation detector 130 may be any radiation detector that detects the radiation and outputs a corresponding signal, for example, a flat panel detector (FPD). The radiation detector 130 may be either a detector of an indirect conversion type, which converts radiation into visible light using a scintillator or the like, and then converts the visible light into an electrical signal using a photosensor or the like or a detector of a direct conversing type, which directly converts the incoming radiation into an electrical signal.

The camera 140 is an example of an optical device that captures an optical image of the object O under the control of the information processing apparatus 200. The camera 140 sends the optical image acquired through imaging to the information processing apparatus 200. The camera 140 may have any known configuration, such as being configured as a video camera capable of capturing moving images or a camera that captures only still images. The camera 140 may be configured to capture images using visible light or using invisible light other than radiation, such as infrared light.

The information processing apparatus 200 includes an optical-image acquisition unit 201, a skeleton estimation unit 202, an object-information determination unit 203, a consistency determination unit 204, a radiographic image acquisition unit 205, an annotation unit 206, a display control unit 207, a human detection unit 208, and an image analysis unit 209. The information processing apparatus 200 further includes a central processing unit (CPU) 231, a storage 232, a main memory 233, an operation unit 234, and a display unit 235. The components of the information processing apparatus 200 are connected together via a CPU bus 230 to mutually send and receive data.

The optical-image acquisition unit 201 can control the camera 140 to acquire an optical image of the object O acquired by the camera 140. The optical-image acquisition unit 201 may acquire an optical image of the object O from the external storage 160 or an optical device (not shown) connected to the information processing apparatus 200 via any network. The optical-image acquisition unit 201 may acquire an optical image stored in the storage 232.

The skeleton estimation unit 202 estimates the human skeleton using the optical image as the input data for a trained model to estimate the skeleton information of the human in the optical image. The trained model that the skeleton estimation unit 202 according to this embodiment uses may be a trained model generated by additionally training a general-purpose trained model for estimating skeleton information, which is obtained using many pieces of training data, using desired data. The desired data may include skeleton information desired by the medical institution or medical front where the radiographic imaging system 100 is used. The detailed process performed by the skeleton estimation unit 202 will be described later.

The object-information determination unit 203 analyzes the estimated skeleton information to determine and recognize object information including information indicating the body part of the object O, which is the target of the radiographic imaging in the optical image, laterality information, and orientational information of the object O. The detailed process performed by the object-information determination unit 203 will be described later.

The consistency determination unit 204 can determine the consistency between the object information determined by the object-information determination unit 203 and the information on the object O included in the radiographic imaging order. The information processing apparatus 200 can support the operator in determining whether the object information obtained using the optical image is consistent with the instruction for radiographic imaging by providing the determination result to the operator.

The radiographic image acquisition unit 205 can control the radiation generation unit 120 and the radiation detector 130 to perform radiographic imaging of the object O, thereby acquiring a radiographic image of the object O from the radiation detector 130. The radiographic image acquisition unit 205 may acquire a radiographic image of the object O from a radiation detector (not shown) connected to the external storage 160 or the information processing apparatus 200 via any network. The radiographic image acquisition unit 205 may acquire a radiographic image stored in the storage 232.

The annotation unit 206 annotates a radiographic image with the object information determined by the object-information determination unit 203. The annotation refers to a process for embedding object information indicating the body part, laterality, and orientation of the object O into the radiographic image. The annotation unit 206 may be configured to annotate an optical image with the object information.

The display control unit 207 can control the display of the display unit 235. For example, the display control unit 207 can display patient information on the object O, imaging conditions, parameters set by the operator, the generated optical image and radiographic image, determined object information, and analysis information on the display unit 235. Examples of the analysis information may include segmentation information. The display control unit 207 can display buttons, sliders, or a graphic user interface (GUI) for receiving operator's operations on the display unit 235.

The CPU 231 is an example of a processor that controls the operation of the information processing apparatus 200. For example, the CPU 231 controls the operation of the entire information processing apparatus 200 according to an operation through the operation unit 234 and the parameters stored in the storage 232 using the main memory 233. The processor for the information processing apparatus 200 is not limited to the CPU. Examples include a microprocessing unit (MPU) and a graphics processing unit (GPU). Other examples of the processor include a digital signal processor (DSP), a data flow processor (DFP), and a neural processing unit (NPU).

The storage 232 can store various images processed by the information processing apparatus 200 and data. The storage 232 may store, for example, patient information, imaging conditions, and parameters set by the operator. The storage 232 may also store, for example, information on a rule-based algorithm for analyzing skeleton information performed by the object-information determination unit 203. Examples of the storage 232 include an optical disk, a memory, and any other storage media. One example of the main memory 233 is a temporary memory for data.

A GPU can perform efficient calculations by processing a larger amount of data in parallel. For this reason, when performing multiple iterations of learning using machine learning algorithms, such as deep learning, processing with a GPU is effective. For this reason, in this embodiment, a GPU may be used in addition to the CPU for the process of the information processing apparatus 200, which serves as an example of a training unit. Specifically, when a training program including a learning model is executed, the training can be performed through calculations carried out cooperatively by the CPU and the GPU. In the processing by the training unit, the calculations may be performed only by the CPU or GPU. The estimation process according to this embodiment may also be implemented using the GPU, similarly to the training unit. If the trained model is provided in an external device, the information processing apparatus 200 may not function as a training unit.

The training unit may include an error detection unit and an update unit (not shown). The error detection unit obtains the error between output data output from the output layer of the neural network and correct answer data according to the input data input to the input layer. The error detection unit may calculate the error between the output data from the neural network and the correct answer data using a loss function. The update unit updates, for example, the node-connection weighting coefficients of the neural network based on the error obtained by the error detection unit to decrease the error. The update unit updates the connection weighting coefficient and so on using a back propagation method. The back propagation method is a method for adjusting parameters such as the node-connection weighting coefficients of each neural network to decrease the error.

Examples of the machine learning model according to this embodiment include the fully convolutional network (FCN) and SegNet.

Examples of a machine learning model for object recognition include the Region CNN (RCNN), fastRCNN, and fasterRCNN. Examples of a machine learning model for object recognition in a per-region basis include the You Only Look Once (YOLO), Single Shot Detector (SSD), and single shot multiBox detector).

The operation unit 234 includes an input device for operating the information processing apparatus 200, for example, a keyboard and a mouse. The operator can use the operation unit 234 in inputting parameters related to a rule-based algorithm for analysis processing using the skeleton information, performed by the object-information determination unit 203.

The display unit 235 includes, for example, any display, and displays various information such as object information and various images under the control of the display control unit 207. The display unit 235 may be, for example, a console monitor for operating the radiographic imaging apparatus including the radiation generation unit 120 and the radiation detector 130. The display unit 235 may be a sub-monitor installed at a position where the operator can observe the sub-monitor even while supporting the positioning of the object O or the console monitor of a radiation emitter. The display unit 235 may be a display that allows the operator to check the display with minimal eye movement, such as a head-mount display that allows the operator to work while wearing it. The display unit 235 may be a touch-panel display, in which case the display unit 235 can also serve as the operation unit 234.

The information processing apparatus 200 may be a computer provided with a processor and a memory. The information processing apparatus 200 may be a general computer or a computer only for the radiographic imaging system. The information processing apparatus 200 may be, for example, a personal computer (PC), a desktop PC, A notebook PC, or a tablet PC (a potable information terminal). The information processing apparatus 200 may be a cloud-based computer in which some components are located in an external device.

The optical-image acquisition unit 201, the skeleton estimation unit 202, the object-information determination unit 203, the consistency determination unit 204, the radiographic image acquisition unit 205, the annotation unit 206, the display control unit 207, the human detection unit 208, and the image analysis unit 209 may be software modules executed by the CPU 231. These components may be circuits that function as specific functions, such as an application specific integrated circuit (ASIC), and independent devices.

Next, the operation of the information processing apparatus 200 according to the control of the CPU 231 will be described. First, the information processing apparatus 200 starts to prepare for imaging based on imaging order information sent from an information management unit (not shown), and the optical-image acquisition unit 201 starts to obtain an optical image under the control of the CPU 231. The imaging order information is information corresponding to a unit of examination ordered by the doctor and includes patient information, imaging (scheduled) date, and the body part, orientation, posture, and so on of the imaging target based on the observation of the doctor. The imaging order information includes information necessary for radiographic imaging, for example, the intended purpose of the radiation detecting apparatus (standing position, supine position, or portable), the patient's posture (imaging part, orientation, etc.), and radiographic imaging conditions (tube voltage, tube current, whether or not there is a grid).

The optical-image acquisition unit 201 acquires an optical image of the object O by controlling the camera 140. The optical image acquired by the optical-image acquisition unit 201 is sequentially transferred to the main memory 233, the skeleton estimation unit 202, the human detection unit 208, and the object-information determination unit 203 via the CPU bus 230.

The skeleton estimation unit 202 uses the transferred optical image as input data for the trained model to estimate the skeleton information of the object O. Next, the object-information determination unit 203 obtains object information from the estimated skeleton information. The object information is transferred to the consistency determination unit 204 via the CPU bus 230. The consistency determination unit 204 compares the imaging order information with the object information and outputs the consistency determination result. The optical image, the skeleton information, the object information, and the consistency determination result are transferred to the storage 232 and the display control unit 207 via the CPU bus 230. The storage 232 stores the various pieces of transferred information. The display control unit 207 displays the various pieces of transferred information on the display unit 235.

The operator checks the various pieces of displayed information and provides operation instructions as necessary via the operation unit 234. For example, if the consistency determination result is correct, the operator provides a radiographic imaging instruction via the operation unit 234. This imaging instruction is sent to the radiographic image acquisition unit 205 by the CPU 231.

In response to receiving the imaging instruction, the radiographic image acquisition unit 205 controls the radiation generation unit 120 and the radiation detector 130 to execute radiographic imaging. In radiographic imaging, first, the radiation generation unit 120 emits a radiation beam toward the object O, and the radiation beam that has passed through the object O while attenuating is detected by the radiation detector 130. The radiographic image acquisition unit 205 obtains a signal corresponding to the intensity of the radiation beam detected by the radiation detector 130 as a radiographic image. The data of the radiographic image is transferred to the main memory 233 and the annotation unit 206 via the CPU bus 230.

The annotation unit 206 annotates the transferred radiographic image with the object information stored in the storage 232. The annotated radiographic image is transferred to the storage 232 and the display control unit 207 via the CPU bus 230. The storage 232 stores the transferred annotated radiographic image. The display control unit 207 displays the transferred annotated radiographic image on the display unit 235. The operator can check the displayed radiographic image and provide a necessary operation instruction via the operation unit 234. The human detection unit 208 uses the optical image transferred from the optical-image acquisition unit 201 as input data for the trained model to detect a human body captured in the optical image. The optical image may include only the object O, or may include multiple human bodies (humans). When multiple humans are included, the human detection unit 208 detects the multiple humans. An example of the case where multiple humans are included is a case where the object O and the radiology technician are included. If the object O cannot take the imaging posture (the posture included in the imaging order information) by himself/herself (for example, the object O is an elderly person or a child), the radiology technician may adjust the imaging posture while supporting the object O. For this reason, the object O and the radiology technician may be captured in the optical image during supporting. The human body includes not only the whole body of the human but also body parts such as the hands, feet, and waist. The detection of the human body may use an existing neural network model.

The information on the detected human body is transferred to the consistency determination unit 204 via the CPU bus 230. The consistency determination unit 204 compares the imaging order information and the object information (the imaging part, laterality, orientation, etc.) and outputs the consistency determination result.

The optical image, the skeleton information, the object information, and the consistency determination result are transferred to the storage 232 and the display control unit 207 via the CPU bus 230.

The storage 232 stores the various pieces of transferred information. The display control unit 207 displays the various pieces of transferred information on the display unit 235.

The image analysis unit 209 analyzes the human body in the optical image, the human body being detected by the human detection unit 208. Since the target of the image analysis is a single human body, if multiple human bodies are detected, image analysis is performed on a human body closest to the radiation exposure field among the multiple human bodies.

### Outline of Image Analysis Unit and Consistency Determination Unit

Thoracoabdominal imaging in general radiography is often performed in the order of capturing the chest first, followed by the abdomen. For this reason, in the case where the imaging part in the imaging order information is only the abdomen, there is a risk of incorrectly capturing an image of the chest when only the abdomen should be imaged. Therefore, the imaging part in the imaging order information is analyzed to determine whether the imaging part in the imaging order information is correctly imaged, and if there is a risk of incorrect imaging, an alert such as “Check: Imaging part” may be displayed on the display unit 235.

A risk of the operator misreading the imaging part may also be anticipated. For example, the lumbar spine (L-SPINE) and the thoracic spine (T-SPINE) can be particularly misread, depending on the size of the characters displayed on the operation screen of the display unit 235. Therefore, the lumbar spine and the thoracic spine may also be analyzed to determine whether the target part is correctly centered within the radiation exposure field, and if there is a risk of incorrect imaging, an alert such as “Check: Imaging part” may be displayed on the display unit 235.

Depending on the imaging part, the image-capturing direction can be incorrect. For example, in chest imaging in standing position, posterior-anterior (PA) imaging to make the heart closer to the radiation detector 130 is often performed, but anterior-posterior (AP) imaging is rarely performed. Similarly, in lateral imaging, right lateral (RL) imaging is often performed, but left lateral (LL) imaging is rarely performed. At that time, there is a risk of imaging in an incorrect direction because of the radiology technician's misconception. Therefore, if there is a risk of imaging in the incorrect direction, an alert such as “Check: Field of View Position” may be displayed on the display unit 235. In addition, the image may be analyzed for right lateral oblique (RLO) imaging and light lateral oblique (LLO) imaging and right lateral decubitus (RLD) imaging and left lateral decubitus (LLD) imaging, and if there is a risk of incorrect imaging, an alert may be displayed on the display unit 235.

Furthermore, in imaging limbic joints with laterality, there is a risk not only of selecting an incorrect imaging part but also of confusing the left and right sides. For example, there is a risk of imaging the right hand although the left hand should be imaged. In imaging the limbic joints with laterality, the orientation may also be identified in addition to the imaging part and laterality. For these instructions, it is determined whether the imaging part, the laterality, and the orientation are correct, and if there is a risk of incorrect imaging, an alert may be displayed on the display unit 235. For example, if the laterality is incorrect among the imaging part, laterality, and orientation, an alert such as “Check: Laterality” may be displayed on the display unit 235. For example, if the laterality and orientation are incorrect among the imaging part, laterality, and orientation, an alert such as “Check: Laterality/Orientation” may be displayed on the display unit 235.

There may be a case where the consistency between the imaging order information and the object information (imaging part, laterality, and orientation) cannot be determined. For example, if a hand other than the imaging target hand appears, an alert such as “Not Determinable” may be displayed on the display unit 235.

if no human body can be detected, an alert such as “patient Undetected” may be displayed on the display unit 235.

### Details of Image Analysis Unit and Consistency Determination Unit

Next, the details of the image analysis unit 209 and the consistency determination unit 204 will be described.

The image analysis unit 209 analyzes the image acquired by the optical-image acquisition unit 201 and transferred from the camera 140.

The images transferred from the camera 140 are sequentially analyzed. If a moving image is acquired, the frames may be thinned out based on the processing load of the image analysis. The analysis process may be performed using a GPU.

The image analysis unit 209 analyzes the posture of the object O by performing skeleton estimation on the human body to be analyzed. The skeleton estimation may be performing using an existing neural network model.

If the camera 140 is installed in the radiation generation unit 120, the camera 140 is moved when the radiation generation unit 120 (or a collimator provided in the radiation generation unit 120) is moved. For example, when the radiation generation unit 120 is rotated by 90°, the camera 140 is also rotated by 90°. As a result, the human body captured in the optical image is also rotated by 90° (the body axis rotates by) 90°.

The accuracy of human detection and skeleton estimation may change when the body axis direction changes. For this reason, for example, human detection is performed with the optical image kept unchanged (rotated by) 0°, and then human detection is performed with the optical image rotated by 90°. Next, human detection is performed with the optical image rotated by 180°, and then human detection is performed with the optical image rotated by 270°. Finally, among the detection results, a result with the highest detection accuracy may be employed.

In radiographic imaging, the radiation generation unit 120 is often rotated at adjustment before imaging, rather than during imaging. For this reason, after the result (rotation angle) with the highest accuracy is employed in the above method, by performing analysis only using the rotation angle until the next imaging, the load of the analysis process can be decreased. Whether the imaging has shifted to the next imaging may be determined depending on whether new imaging order information is obtained or whether the human body has disappeared from the optical image.

Depending on the rotation angle of the radiation generation unit 120, no human body may be detected. In that case, by rotating the optical image at the rotation angles of 0°, 90°, 180°, and 270°, and analyzing the individual images, the human body may be detected. When the human body is detected, the optical image at the detection angle is analyzed.

Of course, when the rotation angle of the radiation generation unit 120 can be obtained, the rotation angle of the optical image to be analyzed may be adjusted based on the obtained rotation angle.

When the rotation angle of the analysis target optical image is to be obtained, the rotation angle, among the multiple optical images captured at different rotation angles, that provides high detection accuracy is stored as a history, and when the history becomes stable (when the rotation angle with the high detection accuracy can be predicted), the optical image may be analyzed only at the rotation angle.

The rotation of the image has been described using four angles: 0°, 90°, 180°, and 270°, but this is illustrative only. For example, the image may be rotated at finer intervals (for example, 45° increments).

If no human body is detected or skeleton estimation cannot be performed even using the above method, an alert such as “Undetected” may be displayed on the display unit 235.

The consistency determination unit 204 obtains an optical center position within the optical image (described later) and performs consistency determination using the center position.

When skeleton estimation is performed, first, consistency determination is performed on the imaging part.

In the determination of the imaging part, it is determined whether the imaging part included in the imaging order information is present in the vicinity of the center of the optical image.

In the determination of the orientation, the orientation is determined based on the positional relationship of the estimated skeleton.

In the determination of the laterality, consistency is determined based on whether a body part corresponding the laterality specified in the imaging order information is located near the center of the optical image. For example, imaging of the hand of the object O may sometimes be supported by the hand of a person other than the object O (the radiology technician's hand). At that time, when it is difficult to make a determination due to the overlap between the object's hand and the radiology technician's hand, an alert such as “Undeterminable” may be displayed on the display unit 235.

The order of determination of the imaging part, determination of the orientation, and determination of the laterality may be any order. Depending on the imaging part, the determination may be started from the orientation or from the laterality.

The orientation or laterality is sometimes not included in the imaging order information. In that case, consistency determination on the orientation or laterality is not performed.

Even when the orientation is not included in the imaging order information, the determination may be determined based on another information. For example, if the imaging technique included in the imaging order information is “Rosenberg method” for knee radiography, the orientation may be determined.

Thus, if there is a risk of incorrect imaging in the actual imaging part, orientation, and laterality of the object relative to the imaging order information, an alert is displayed on the display unit 235. This enables the radiology technician to check whether the imaging position of the object is suitable before imaging.

When an alert is displayed, the radiation generation unit 120 may be controlled to disable or enable the application of radiation. Since imaging is possible even if the information processing apparatus displays an alert, it is useful to control the radiation generation unit 120 so as to execute radiation exposure.

### Related Example 1

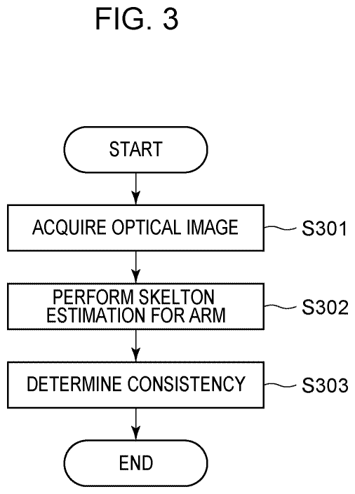

Referring to FIGS. 3 to 7, a radiographic imaging system, an information processing apparatus, and a method for information processing according to Related Example 1 will be described. Related Example 1 describes a process for recognizing the laterality of the object to be imaged from optical images acquired at a predetermined frame rate using a video camera as the camera 140 and outputting the laterality as object information. The laterality refers to information indicating whether the body part with left-right symmetry, intended for imaging, is on the left or the right side.

### Processing Procedure

A series of processing steps according to Related Example 1 will be described hereinbelow with reference to FIG. 3. FIG. 3 is a flowchart showing the processing steps according to Related Example 1. When the processing steps according to Related Example 1 are started, the process proceeds to step S301.

### Step S301

In step S301, the optical-image acquisition unit 201 controls the camera 140 to acquire an optical image of a radiographic imaging part including the object of radiographic imaging. In Related Example 1, the camera 140 is a video camera attached to the radiation generator. The camera 140 captures images of the object who takes an imaging posture on the radiation detector 130 placed on a supine table and outputs optical images at predetermined frame rate.

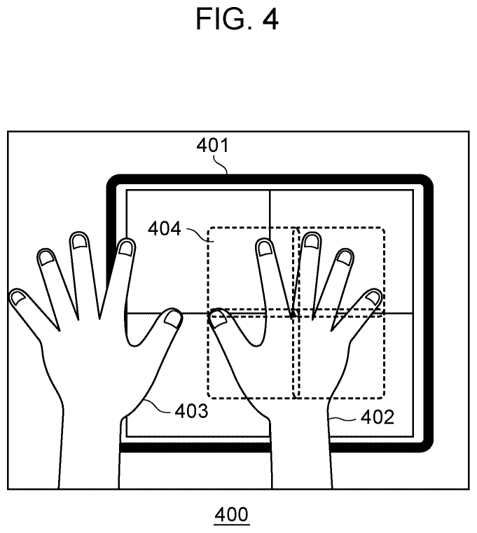

Here, an example in which the radiation detector 130, a right hand 402, and a left hand 403 appear in an optical image 400 will be described with reference to FIG. 4. FIG. 4 illustrates an example of the optical images according to Related Example 1. A radiation detector field 401 in the optical image 400 is a field that represents the radiation detector 130. In the example illustrated in FIG. 4, the light of a collimator lamp is applied onto the right hand 402 of the object, and a collimator lamp exposure field 404 is visualized above the right hand 402 in the optical image 400. The collimator lamp is a device to radiate visible light, the collimator lamp being attached to the collimator, to confirm a radiation target field (where radiation is to be emitted) before radiation is emitted. The collimator lamp exposure field 404 generated by the collimator lamp coincides with the radiation target field during radiographic imaging.

### Step S302

In step S302, the skeleton estimation unit 202 estimates the skeleton information of the object by using the optical image acquired by the optical-image acquisition unit 201 as input data for the trained model. Here, the skeleton information refers to information that indicates multiple predefined feature points on the object, including coordinates indicating body parts such as the face, shoulders, hands, waist, and feet and is used to recognize the posture of the human body. In Related Example 1, the skeleton estimation unit 202 uses a trained model composed of a machine learning model, such as neural networks. The trained model is obtained by training a model that is generated in advance through supervised machine learning using a variety of image data not limited to radiographic imaging using additional data corresponding to outputs desired in radiographic imaging parts.

The feature points may include, regarding the head, at least one of the eyes, nose, and mouth, to enable the distinction between the front and back of the head. Regarding the body, the feature points may include feature points to enable the distinction among, for example, the neck, chest (thoracic spine), abdomen, and waist (lumbar spine). Regarding the limbs, the feature points may include, for example, the shoulders, elbows, wrist, hip joint, knees, and ankles. Since the skeleton estimation unit 202 estimates skeleton information that indicates these feature points, the information processing apparatus 200 can recognize a broad range of posture. For parts with laterality, such as the right hand or the left hand, the feature points may be defined as feature points that differ depending on the skeleton estimation. In this case, the information processing apparatus 200 may estimate the laterality based on the feature points.

The skeleton estimation process may be executed on optical images using a single neural network model but may include an object-part extraction process for extracting an object region from an optical image and may be configured to perform skeleton estimation on the extracted object region. In this case, the object region in the optical image may be used as input data for the trained model. The object region in the optical image may be used for training data. The skeleton estimation process may include a classification process for determining whether the whole body of the object or a part such as the head, hand, or foot appears in the optical image. In this case, the skeleton estimation unit 202 may select and apply a class-specific trained model for skeleton estimation specialized in the whole body, head, arm, or foot based on the result of the classification process.

A trained model designed for generic purposes, trained using a large volume of data including optical images and skeleton information of the object in the optical images, is generally used for a neural network model for skeleton estimation. However, when such a trained model is used as a skeleton estimation unit tailored to the requirements of medical fronts, which is the target of Related Example 1, for example, outgoing feature points may fall short. For example, regarding the four limbs, the trained model may be trained to output feature points such as the shoulder joint, elbow joint, wrist, hip joint, knee joint, and ankle, but it might not be capable of outputting feature points for fingers, toes, or additional points indicating the front and back of these parts. For this reason, in Related Example 1, a trained model that is additionally trained based on such a general-purpose trained model is used so as to output feature points required for each medical institution or medical front. Such additional training of a neural network model for additionally outputting feature points for some human body parts is achieved by machine learning using a relatively smaller data set compared to the data set used for training general-purpose trained models. For example, additional training may be performed using a data set that is necessary and sufficient to output feature points necessary for each medical institution or medical front in which the radiographic imaging system 100 is used.

Training data for additional training may include a data set in which an optical image is used as input data, and data or images with labels indicating feature points that require additional training in the optical image are used as output data (correct answer data). The correct answer data may be generated by doctors or the like based on the optical image. A trained model trained using such training data can output, when an optical image is input, the coordinates of individual feature points including the additionally trained feature point in the input optical image and probability indicating the likelihood of being the feature point. If the probability is 0.0, the trained model may not output the position of the feature point. The training data of a general-purpose trained model before undergoing additional training may also include a data set of a similar format, in which case, the training data for use in additional training include skeleton information on a skeleton different from the skeleton indicated by the skeleton information that the general-purpose trained model learned. The trained model for the skeleton estimation unit 202 may have already undergone additional training and does not have to be trained for each process.

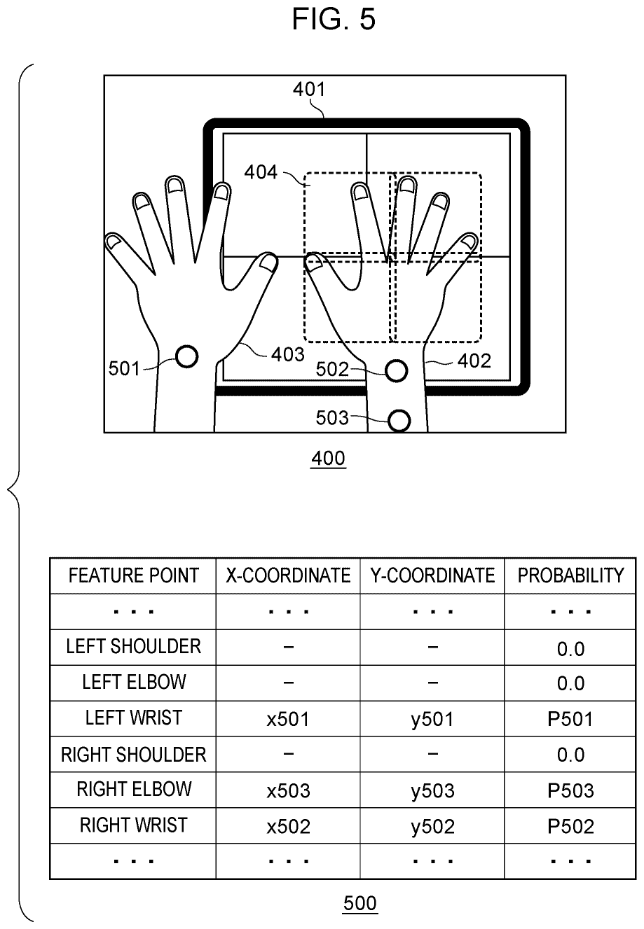

In Related Example 1, the skeleton estimation unit 202 performs arm skeleton estimation in which the left wrist, left elbow joint, left shoulder joint, right wrist, right elbow joint, and right shoulder joint are estimated as skeleton information, and the arm skeleton estimation is applied to the optical image 400. In this example, the skeleton estimation unit 202 inputs the optical image 400 to the trained model. In this case, the trained model detects a left wrist 501 and a right wrist 502 and outputs the respective coordinates (x501, y501) and (x502, y502) and probabilities P501 and P502 representing their respective likelihoods of being feature points, as shown in FIG. 5. In contrast, the trained model estimates the probability that the left shoulder joint, the left elbow joint, and the right shoulder joint appear in the optical image 400 is 0.0 and does not output corresponding coordinates. In contrast, the trained model estimates that a right elbow joint 503 is present at coordinates (x503, y503) at a probability of P503. The estimation regarding the right elbow joint is incorrect, and in this case, it is expected that the probability P503 may be smaller than probabilities P501 and P502.

The skeleton estimation unit 202 outputs, as skeleton information, the coordinates and probabilities of the individual feature points in a format like Table 500. The skeleton estimation unit 202 may output the skeleton information in a format according to the output of the trained model. For example, the trained model may output matrix data in the form of Table 500, and the skeleton estimation unit 202 may output the matrix data or in the form of Table 500. For example, the trained model may output a map (heat map) in which the feature amounts extracted from the optical image are visualized. In this case, the skeleton estimation unit 202 may output the map or the position and probability of the feature point with the highest probability among the feature points in the map in the form of Table 500.

### Step S303

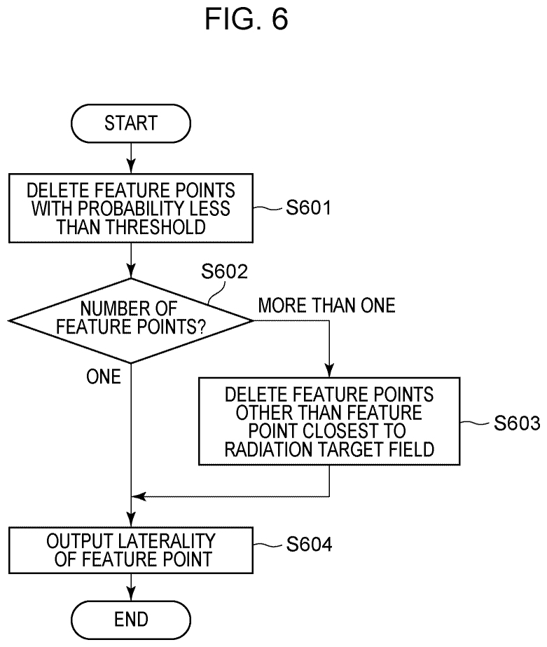

In step S303, the object-information determination unit 203 determines the laterality of the target object of radiographic imaging from the skeleton information estimated by the skeleton estimation unit 202 and outputs the laterality as object information. Specifically, the object-information determination unit 203 applies the rule-based algorithm as shown in FIG. 6 to the skeleton information estimated by the skeleton estimation unit 202. In the rule-based algorithm, the laterality of the imaging target object is determined based on the probability and coordinates of the skeleton information, which is the output of the skeleton estimation unit 202.

Here, the description is made using an example in which the rule-based algorithm in FIG. 6 is applied to the output of skeleton estimation shown in FIG. 5. The output of skeleton estimation shown in FIG. 5 includes the left wrist 501, the right wrist 502, and the right elbow joint 503. The probability P503 of the right elbow joint 503 is small.

First, in step S601, the object-information determination unit 203 deletes feature points whose probability is less than a threshold from the feature points included in the skeleton information output by the skeleton estimation unit 202. In the above example, the object-information determination unit 203 determines that the probability P503 of the right elbow joint 503 is less than the threshold and deletes the right elbow joint 503, which is a feature point.

Next, in step S602, the object-information determination unit 203 determines whether the number of remaining feature points is one or more than one. If in step S602 it is determined that the number of feature points is more than one, the process proceeds to step S603. In contrast, if in step S602 it is determined that the number of feature points is one, the process proceeds to step S604. In the above example, since the object-information determination unit 203 determines that the number of remaining feature points is two, the left wrist 501 and the right wrist 502, the process proceeds to step S603.

In step S603, the object-information determination unit 203 determines the position of a radiation target field where radiation is to be emitted (radiation exposure field) in the optical image and deletes feature points other than the feature point closest to the radiation target field from among the remaining feature points. In the above example, the object-information determination unit 203 calculates the distance between the radiation target field and the coordinates (x501, y501) of the left wrist and the distance between the radiation target field and the coordinates (x502, y502) of the right wrist.

Here, the position of the radiation target field in the optical image 400 may coincide with the position of the collimator lamp exposure field 404. Therefore, the object-information determination unit 203 may extract the collimator lamp exposure field 404 through a threshold process based on, for example, the luminance of the optical image and calculate the distance between the collimator lamp exposure field 404 regarded as the radiation target field and the feature point. The method for extracting the collimator lamp exposure field 404 in the optical image is illustrative only; any other known method may be employed. For example, the object-information determination unit 203 may extract the collimator lamp exposure field 404 using edge detection or corner detection.

The distance dn between the coordinates (xn, yn) of a feature point n and the representative point (for example, the center position (xc, yc)) of the radiation target field can be calculated according to Eq. 1.

$$dn = \sqrt{{( xc - xn )}^{2} + {( yc - yn )}^{2}} \qquad ( Eq . 1 )$$

The object-information determination unit 203 can compare the distances between the individual feature points and the radiation target field, calculated according to Eq. 1, to determine a feature point closest to the radiation target field and delete feature points other than the feature point. In the above example, among the outputs of the skeleton estimation shown in FIG. 5, the right wrist 502 is closest to the radiation target field, and therefore, the object-information determination unit 203 deletes the feature point of the left wrist 501.

In step S604, the object-information determination unit 203 outputs the laterality of the remaining feature point as object information. In the above example, the object-information determination unit 203 outputs the laterality “right” of the remaining feature point of the right wrist 502. When in step S604 the laterality of the feature point is output, the laterality determination process, which is the object-information determination process according to Related Example 1, ends.

In the object-information determination process, the correspondence relationship between the remaining feature point and the output feature point may be predetermined by a rule. For example, if a feature point, the right elbow joint or the right wrist, remains, the laterality “right” is output, and if a feature point, the left elbow joint or the left wrist, remains, the laterality “left” is output.

The above description pertains to the case where the feature points are the right wrist and the left wrist; however, other feature points such as the shoulder joints, the elbow joints, the hip joints, the knee joints, or the ankles may also be used. Even in this case, the object-information determination unit 203 can output, as object information, the laterality of the feature point included in the skeleton information output by the skeleton estimation unit 202.

The rule in the object-information determination process is not limited to the above rule. For example, in the object-information determination process, if the laterality to be determined is laterality regarding “hand” when the remaining feature point is “right shoulder joint” or “left shoulder joint”, it can be considered that some errors have occurred. For this reason, a rule that an error message is output is conceivable.

The radiation target field is not limited to the collimator lamp exposure field and may be determined using another known method. For example, a simpler approach may involve using the center of the optical image as the radiation target field. In this case, the object-information determination unit 203 may output the laterality of a feature point close to the center of the optical image as the object information. In this case, even when the collimator lamp exposure field does not appear in the optical image, the laterality of the object can be determined. For example, a marker or another sign that indicates the radiation target field may be provided on the imaging table, and the marker in the optical image is extracted by the object-information determination unit 203 to determine the radiation target field.

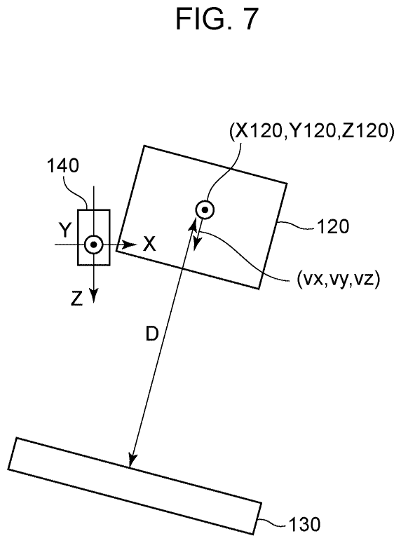

If higher accuracy is required in the determination of object information, the radiation target field in the image may be calculated in consideration of the spatial positions of the radiation generation unit 120 and the camera 140. For example, as shown in FIG. 7, assume a camera coordinate system in which the optical center of the camera 140 is defined as the origin (0, 0, 0), the optical axis direction of the camera 140 as the Z-axis, the horizonal direction of the image as the X-axis, and the vertical direction as the Y-axis. Here, assume a case where the spatial coordinates (X120, Y120, Z120) of the radiation generation unit 120 and the radiation emission direction (vx, vy, vz) are known.

These information can be obtained by attaching the camera 140 to the radiation generation unit 120 while measuring the position with a measure. The information may be obtained based on, for example, the amount of drive from the location of the radiation generation unit 120 using a device capable of mechanically measuring the amount of drive, such as a stepping motor. The information may be obtained by attaching a gyro mechanism, an acceleration sensor, or a similar device to the radiation generation unit 120 to acquire the displacement in position or angle from the initial position, and inputting this data into the information processing apparatus 200. When the camera 140 is attached to the radiation generation unit 120, the spatial coordinates (X120, Y120, Z120) and the radiation emission direction (vx, vy, vz) of the radiation generation unit 120 in the camera coordinate system after the attachment may be generally fixed values. In this case, the determined coordinates (xp, yp) of the radiation target field in the optical image can be expressed by Eq. 2.

$$xp = fx \cdot \frac{X 120 + vx \cdot D}{Z 120 + vz \cdot D} + cx , yp = fy \cdot \frac{Y 120 + vy \cdot D}{Z 120 + vz \cdot D} + cy \qquad ( Eq . 2 )$$

where D is the distance between the radiation generation unit 120 and the radiation detector 130, (fx, fy) is the focal length of the camera 140 and (cx, cy) is the optical center of the camera 140.

By determining the radiation target field (xp, yp) as described above, the object-information determination unit 203 can determine the laterality of the object more accurately by outputting the laterality of a feature point closest to the radiation target field (xp, yp) as the object information.

### Application Example of Determined Object Information

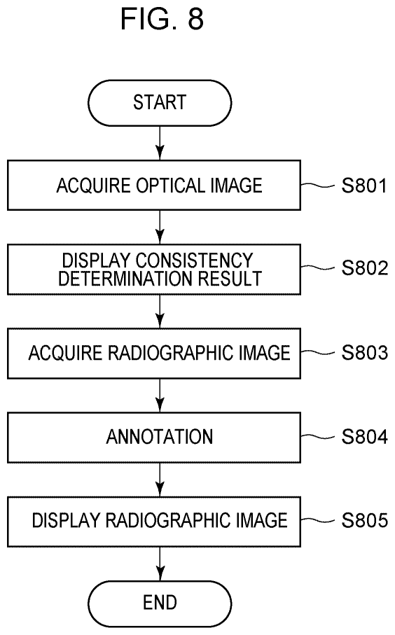

One example of a process using the determined object information will be described with reference to FIG. 8. In this example, when a process using the object information is started, the object information determined by the object-information determination process is transferred to the consistency determination unit 204 via the CPU bus 230, and the process proceeds to step S801. In step S801, the consistency determination unit 204 determines the consistency between imaging order information and the object information. If the laterality included in the obtained object information is “right” when the imaging target region and the laterality in the imaging order are “hand” and “right”, respectively, the consistency determination unit 204 outputs “consistent”, and in all other cases, outputs “inconsistent”.

In step S802, the display control unit 207 displays the optical image acquired by the optical-image acquisition unit 201, the object information output from the object-information determination unit 203, the imaging order, and the consistency determination result on the display unit 235. The display control unit 207 can also display the object region and skeleton information obtained by the skeleton estimation unit 202 and analysis information obtained during the object-information determination process on the display unit 235.

In step S803, the operator checks the information displayed on the display unit 235 and then inputs a radiographic image imaging instruction to the information processing apparatus 200 via the operation unit 234. The radiographic image acquisition unit 205 acquires a radiographic image in response to the radiographic image imaging instruction from the operator. Specifically, the radiographic image acquisition unit 205 causes the radiation generation unit 120 to emit a radiation beam on the imaging conditions corresponding to the imaging order and acquires a radiographic image from the radiation detector 130 that has detected radiation transmitted through the object O. The radiographic image acquisition unit 205 may acquire the radiographic image in response to the completion of the consistency determination process. In this case, the radiographic image acquisition unit 205 may acquire a radiographic image corresponding to the optical image used in the object-information determination process from the external storage 160 or the storage 232.

In step S804, the object information is transferred to the annotation unit 206, for example, via the CPU bus 230, and the annotation unit 206 annotates the acquired radiographic image with the object information. For example, since the radiographic image is a transparent image, it is difficult to determine whether the hand in a radiographic image of the right hand is the right hand or the left hand. However, by the annotation unit 206 placing the laterality information, such as “right”, as annotation information in the radiographic image, the information that the object in the radiographic image is the right hand can be added to the radiographic image. The radiographic image annotated with the object information can be transferred to the display control unit 207 or the storage 232 via the CPU bus 230. The annotation unit 206 may add the object information to the optical image, in which case, the optical image annotated with the object information can be transferred to the display control unit 207 or the storage 232 via the CPU bus 230.

In step S805, the display control unit 207 can display the radiographic image annotated with the object information on the display unit 235. The display control unit 207 may display the optical image and the radiographic image annotated with the annotated object information on the display unit 235 either side by side or switching between them. When the radiographic image display process is completed, the processing using the object information ends. The display unit 235 may display the optical image annotated with the object information.

Thus, the radiographic imaging system 100 according to Related Example 1 includes the radiation generation unit 120 and the radiation detector 130, which perform radiographic imaging of the object, the camera 140 that functions as an example of an optical device that captures an optical image of the object, and the information processing apparatus 200. The information processing apparatus 200 includes the optical-image acquisition unit 201, the skeleton estimation unit 202, and the object-information determination unit 203. The optical-image acquisition unit 201 functions as an example of an acquisition unit that acquires an optical image of the object in a radiographic imaging part. The skeleton estimation unit 202 functions as an example of an estimation unit that estimates skeleton information of the object in the acquired optical image by using the acquired optical image as input data for a second trained model obtained by additionally training a general-purpose trained model (a first trained model) for estimating skeleton information on the object's skeleton using skeleton information on a skeleton different from the skeleton that the skeleton information of the first trained model learned. The object-information determination unit 203 functions as an example of a determination unit that determines object information including the laterality information of the object in the optical image using the skeleton information of the object in the optical image.

With the above configuration, the information processing apparatus 200 according to Related Example 1 can determine the laterality of the object (the target of radiographic imaging) as object information using the skeleton information estimated from the optical image. This enables the information processing apparatus 200 to appropriately support the operator to determine whether the laterality of the object is correct using the determined object information. The determined object information may also be used as additional information to be provided to the operator in addition to a live image in radiographic imaging for medical examinations. The trained model for use in estimating skeleton information according to Related Example 1 is obtained by performing additional training tailored to the objectives of each medical institution or medical front on the general-purpose trained model. This therefore enables the information processing apparatus 200 to appropriately estimate skeleton information required in medical institutions or medical fronts without the need for collecting a large amount of training data.

The object-information determination unit 203 can determine object information through rule-based processing. This enables the information processing apparatus 200 to determine object information through relatively easily adjustable rule-based processing without the need to adjust a trained model, which is difficult to modify in terms of configuration. Therefore, the information processing apparatus 200 can determine object information using rules according to the operation and knowledge in medical institutions or medical fronts.

The skeleton estimation unit 202 can estimate, as the skeleton information of the object in the optical image, the coordinates of multiple feature points of the object in an optical image and the probability that the multiple feature points correspond to the feature points of the object. In this case, the object-information determination unit 203 can select a feature point of the object in the optical image using a threshold of the probability estimated by the skeleton estimation unit 202 and determine object information using the selected feature point. The information processing apparatus 200 can determine the object information based on a more suitable feature point by selecting feature points in this manner.

The object-information determination unit 203 can determine a radiation target field (radiation exposure field) where radiation is to be emitted in the optical image. The object-information determination unit 203 can select a feature point of the object in the optical image based on the positional relationship between the coordinates estimated by the skeleton estimation unit 202 and the radiation exposure field and determine the object information using the selected feature point. The information processing apparatus 200 can determine the object information based on a more suitable feature point through such selection of the feature point.

The object-information determination unit 203 can determine the radiation exposure field based on the collimator lamp exposure field in the optical image or the center of the optical image. The object-information determination unit 203 can determine the radiation exposure field based on the arrangement of the radiation generation unit 120 that emits radiation and the camera 140, which functions as an example of the optical device that generates an optical image. This allows for an appropriate analysis process according to the location of the device of each medical front by taking account of the position of the radiation generation unit 120 in the camera coordinate system, improving the analysis accuracy of the object-information determination unit 203.

The skeleton estimation unit 202 can extract an object region in the optical image and estimate the skeleton information of the object in the optical image using the object region in the optical image as input data for the second trained model. The skeleton estimation unit 202 can classify the optical image based on whether the whole body, the head, the hand, or the foot of the object appears and can select and use a second trained model corresponding to the classification. In this case, the skeleton estimation unit 202 is expected to estimate more suitable skeleton information.

The second trained model may be obtained by additional training using a smaller number of training data than the number of training data of the first trained model. For example, the second trained model may be obtained by additionally training the first trained model using skeleton information on at least one of feature points of the left and right elbow joints, wrists, fingers, front and back of the hands, knee joints, ankles, toes, and front and back of the feet.

The information processing apparatus 200 may further include the consistency determination unit 204 that functions as an example of a determination unit that determines whether consistency is provided between the object information and information included in a radiographic imaging order. In this case, the information processing apparatus 200 may more suitably support the determination of the operator whether the imaging conditions such as the posture of the object correspond to the imaging order by presenting the determination result by the consistency determination unit 204.

In this context, the information processing apparatus 200 may further include the display control unit 207 that displays at least one of an optical image, object skeleton information in the optical image, an object region in the optical image, object information, and the determination result by the consistency determination unit 204 on the display unit 235. If a determination result indicating inconsistency is output by the consistency determination unit 204, the display control unit 207 can display an alert on the display unit 235. In this case, the information processing apparatus 200 can present the inconsistency to the operator, prompting the operator to make the imaging conditions, such as the posture of the object, consistent with the imaging order, thereby providing more appropriate support for radiographic imaging.

The information processing apparatus 200 may further include the annotation unit 206 that adds the object information as annotation information to the optical image or the radiographic image. In this case, the information processing apparatus 200 can more appropriately support the determination of the operator about the imaging conditions, such as the posture of the object, by presenting the optical image or the radiographic image in which the annotation information is added. The display control unit 207 can display annotation information and an optical image or a radiographic image in which the annotation information is placed on the display unit 235.

### Related Example 2

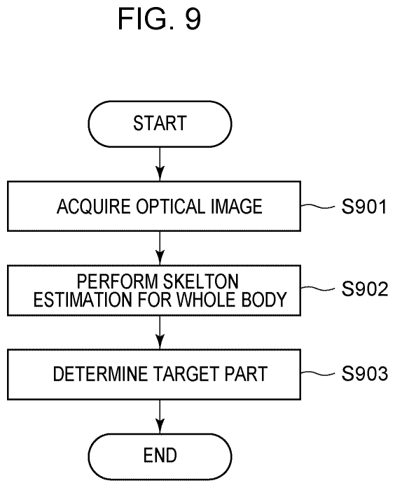

Referring to FIGS. 9 to 14, a radiographic imaging system, an information processing apparatus, and a method for information processing according to Related Example 2 will be described. Related Example 2 describes a process for recognizing the body part of the object to be imaged from optical images acquired at a predetermined frame rate using a video camera as the camera 140 and outputting the recognition result as object information. Examples of the body part of the object include the head, chest, and limbs of the human body. The subdivision level of body parts to be recognized may be varied depending on the intended purpose. For example, the limb may be further divided into finer parts such as the upper arm, elbow, wrist, hand, and fingers. Here, an example will be described in which it is determined whether the imaging target region in the imaging order is “chest” or “abdomen”. Since the configuration of the radiographic imaging system and the information processing apparatus according to Related Example 2 is the same as the configuration of the radiographic imaging system and the information processing apparatus according to Related Example 1, the description will be omitted using the same reference signs. The description of processing similar to that detailed in Related Example 1 will be omitted.

### Processing Procedure

A series of processing steps according to Related Example 2 will be described hereinbelow with reference to FIG. 9. FIG. 9 is a flowchart showing the processing steps according to Related Example 2. When the processing steps according to Related Example 2 are started, the process proceeds to step S901.

### Step S901

In step S901, the optical-image acquisition unit 201 controls the camera 140 to acquire an optical image of a radiographic imaging part including the object of radiographic imaging. In Related Example 2, the camera 140 is a video camera attached to the radiation generator. The camera 140 captures images of the object who takes an imaging posture on the radiation detector 130 placed on a supine table and outputs optical images at predetermined frame rate.

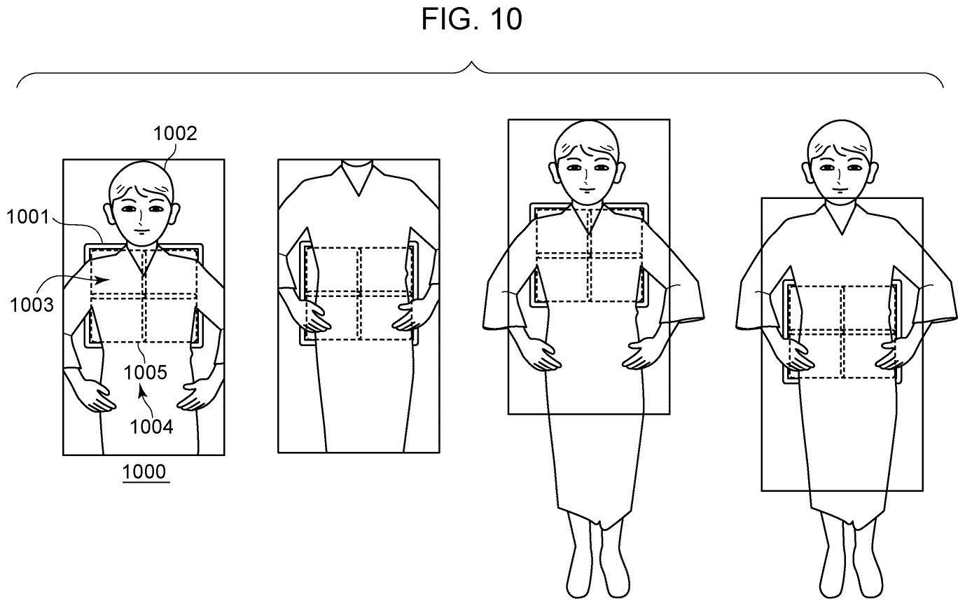

Here, an example in which the radiation detector 130, a head 1002, a chest 1003, and an abdomen 1004 appear in an optical image 1000 will be described with reference to FIG. 10. FIG. 10 illustrates an example of the optical images according to Related Example 2. A radiation detector field 1001 in the optical image 1000 is a field that represents the radiation detector 130. In the example illustrated in FIG. 10, the light of a collimator lamp is applied onto the chest 1003 of the object, and a collimator lamp exposure field 1005 is visualized above the chest 1003 in the optical image 1000. The collimator lamp exposure field 1005 coincides with the radiation target field during radiographic imaging.

### Step S902

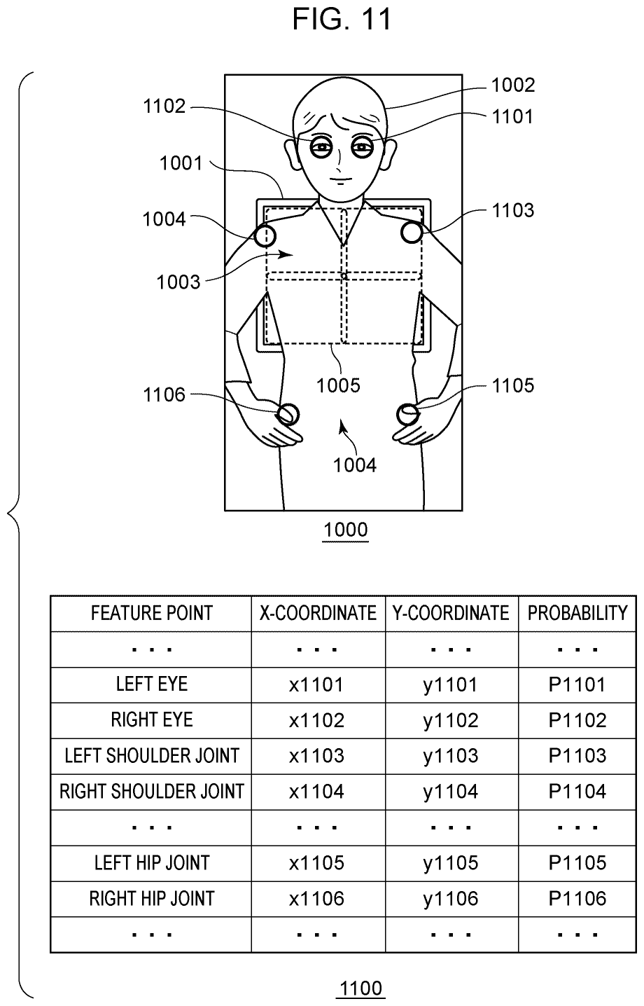

In step S902, the skeleton estimation unit 202 estimates the skeleton information of the object by using the optical image acquired by the optical-image acquisition unit 201 as input data for the trained model. In Related Example 2, the skeleton estimation unit 202 performs whole-body skeleton estimation in which the left and right eyes, left and right shoulder joints, left and right hip joints, left and right elbows, left and right wrists, left and right knees, and left and right ankles are estimated as skeleton information, and the whole-body skeleton estimation is applied to the optical image 1000. The skeleton estimation unit 202 detects left and right eyes 1101 and 1102, left and right shoulder joints 1103 and 1104, and left and right hip joints 1105 and 1106 in the optical image 1000 as feature points, as illustrated in FIG. 11.

The skeleton estimation unit 202 outputs, as skeleton information, the coordinates and probabilities of the individual feature points in a format like Table 1100. Here, it is assumed that the skeleton estimation unit 202 output the coordinates (x1101, y1101) to (x1106, y1106) of the feature points and the probabilities P1101 to P1106 indicating the likelihood of being the feature points. However, the skeleton estimation unit 202 estimated the probability that other skeleton information is included in the optical image 1000 is 0.0 and did not output corresponding coordinates. Unlike Related Example 1, it is assumed that incorrect estimation was not performed for ease of explanation. The skeleton estimation unit 202 may output the skeleton information in a format corresponding to the output of the trained model, similarly to the skeleton estimation unit 202 according to Related Example 1.

The trained model for use in Related Example 2 may also be a trained model obtained by additionally training a trained model for use in general-purpose skeleton estimation so as to output a feature point for each medical institution or medical front, similarly to the trained model according to Related Example 1. The training data may be prepared in advance, similarly to the training data according to Related Example 1.

### Step S903

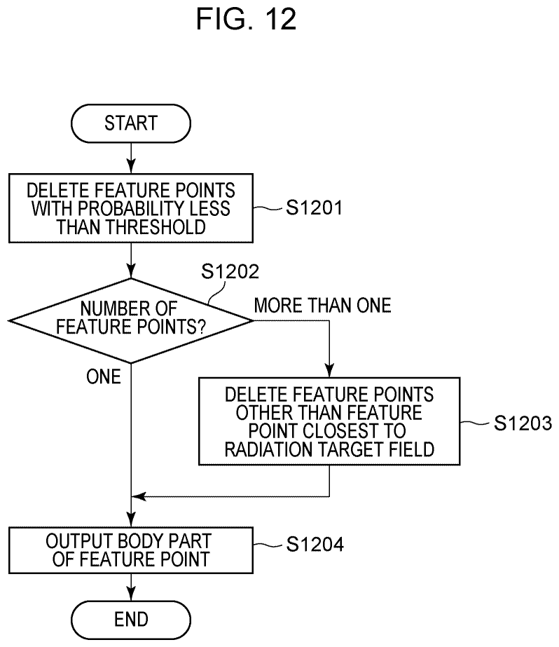

In step S903, the object-information determination unit 203 determines the body part of the target object of radiographic imaging from the skeleton information estimated by the skeleton estimation unit 202 and outputs the body part as object information. Specifically, the object-information determination unit 203 applies the rule-based algorithm as shown in FIG. 12 to the skeleton information estimated by the skeleton estimation unit 202. In the rule-based algorithm, the body part of the imaging target object is determined based on the probability and coordinates of the skeleton information, which is the output of the skeleton estimation unit 202.

Here, the description is made using an example in which it is determined whether the body part of the object is the chest or abdomen by applying the rule-based algorithm in FIG. 12 to the output of skeleton estimation shown in FIG. 11. The output of skeleton estimation shown in FIG. 11 includes the left and right eyes 1101 and 1102, the left and right shoulder joints 1103 and 1104, and the left and right hip joints 1105 and 1106, and their probabilities P1101 to P1106 are equal to or greater than a predetermined threshold.

First, in step S1201, the object-information determination unit 203 deletes feature points whose probability is less than a threshold from the feature points included in the skeleton information output by the skeleton estimation unit 202. In the above example, since all of the probabilities P1101 to P1106 are equal to or greater than a predetermined threshold, no feature points are eliminated as feature points whose probability falls below the threshold.

Next, in step S1202, the object-information determination unit 203 determines whether the number of remaining feature points is one or more than one. If in step S1202 it is determined that the number of feature points is more than one, the process proceeds to step S1203. In contrast, if in step S1202 it is determined that the number of feature points is one, the process proceeds to step S1204. In the above example, since the object-information determination unit 203 determines that the number of remaining feature points is more than one, the process proceeds to step S1203.

In step S1203, the object-information determination unit 203 determines the position of a radiation target field in the optical image and deletes feature points other than the feature point closest to the radiation target field from among the remaining feature points. In the above example, the object-information determination unit 203 calculates the distance between the radiation target field and the coordinates of the feature points (x1101, y1101) to (x1106, y1106).

Here, the position of the radiation target field in the optical image 1000 may coincide with the position of the collimator lamp exposure field 1005. Therefore, the object-information determination unit 203 may extract the collimator lamp exposure field 1005 through a threshold process based on, for example, the luminance of the optical image and calculate the distance between the collimator lamp exposure field 1005 regarded as the radiation target field and the feature point, as in Related Example 1. The radiation target field may be calculated in consideration of the center of the optical image or the spatial positions of the radiation generation unit 120 and the camera 140, as described in as in Related Example 1.

The object-information determination unit 203 can compare the distances between the individual feature points and the radiation target field to determine a feature point closest to the radiation target field and delete feature points other than the feature point. In the above example, among the outputs of the skeleton estimation shown in FIG. 11, either of the left and right shoulder joints 1103 and 1104 is closest to the radiation target field, and therefore, the object-information determination unit 203 deletes the other feature points.

In step S1204, the object-information determination unit 203 outputs the body part of the remaining feature point as object information. In the above example, the object-information determination unit 203 outputs the body part corresponding to either of the left and right shoulder joints 1103 and 1104, which is the remaining feature point. The rule-based algorithm in Related Example 2 is an algorithm for determining whether the body part is the chest or the abdomen, the output determination result is the “chest” close to the shoulder joints. In the algorithm for determining whether the body part is the chest or the abdomen, even if the feature point closest to the radiation target field is either of the left and right eyes, “chest” is output. In contrast, “abdomen” is output when the feature point closest to the radiation target field is the hip joint 1105 or 1106, the left or right wrist, or the left or right knee joint (not shown). When in step S1204 the body part of the object is output, the object-part determination process, which is the object-information determination process according to Related Example 2, ends.

The above description pertains to the case where the feature points are the left and right eyes, shoulder joints, and hip joints, and it is determined whether the body part of the object is the chest or the abdomen. However, other feature points or other body parts such as the head or limbs may also be used in the process of the object-information determination unit 203 for outputting the body part as object information. The rule-based algorithm for determining object information may be varied depending on the difference in the imaging posture employed by the medical institution or medical front or the feature point captured within the angle of view of the camera 140 used. For example, in a rule-based algorithm for determining the body part including the head and limbs, when the feature point closest to the radiation target field is either of the left and right eyes, “head” may be output. In a rule-based algorithm for determining the body part including the left and right wrists and knee joints, the body part in the subdivision level, “left wrist”, “right wrist”, “left knee joint”, or “right knee joint”, may be output.

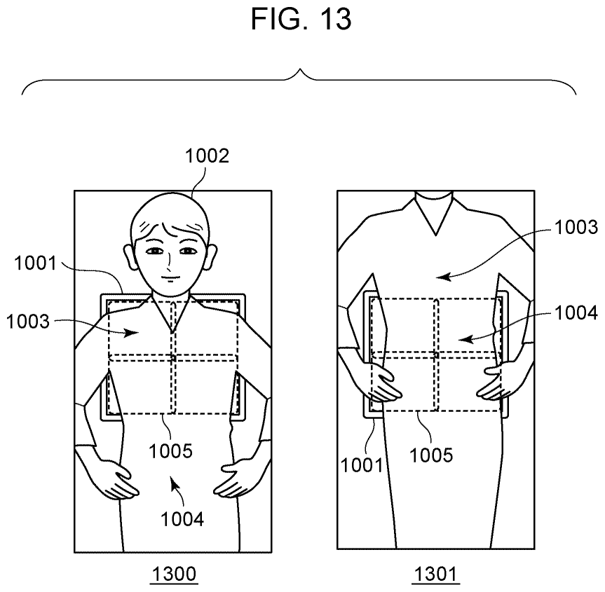

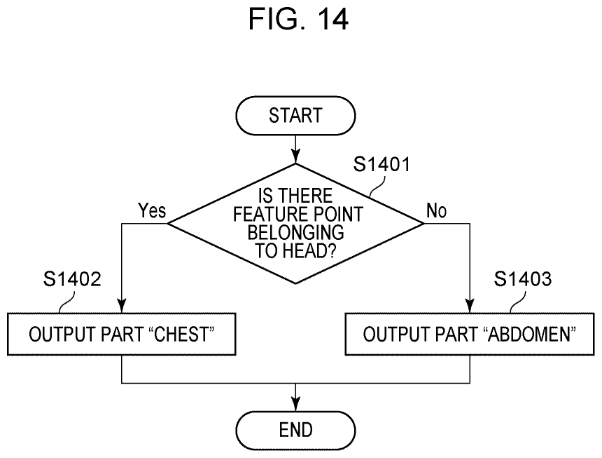

Other rule-based algorithms for determining the body part of the object may also be employed. For example, as shown in FIG. 13, assume that, in a certain medical institution, the camera 140 is installed such that an optical image 1300 is used for thoracic imaging and an optical image 1301 is used for abdominal imaging. In this case, the object-information determination unit 203 may apply the rule-based algorithm as shown in FIG. 14 to the output of skeleton estimation.

In the rule-based algorithm shown in FIG. 14, in step S1401, the object-information determination unit 203 simply determines whether a feature point that belongs to the head is present. If in step S1401 it is determined that a feature point that belongs to the head is present, the process proceeds to step S1402. In step S1402, the object-information determination unit 203 outputs “chest” as object information. In contrast, if in step S1401 it is determined that a feature point that belongs to the head is not present, the process proceeds to step S1403. In step S1403, the object-information determination unit 203 outputs “abdomen” as object information. If in step S1402 or step S1403 the body part of the object is output, the object-part determination process, which is the object-information determination process according to Related Example 2, ends.

Thus, the rule-based algorithm for determining object information may be adapted to the application of the medical institution or medical front where the radiographic imaging system 100 and the information processing apparatus 200 are used. Examples of the application to the medical institutions or medical front may include rules regarding the posture of the object and the placement of the camera 140 during radiographic imaging according to the imaging part.

### Application Examples of Determined Object Information

The object information including the body part of the object obtained above may be used for annotation or similar processes, as in Related Example 1. For example, the object information including the body part of the object obtained may be transferred to the consistency determination unit 204 via the CPU bus 230 and may be used for consistency determination between the imaging order information and the object information. The display control unit 207 may display the object information and analysis information obtained in the process on the display unit 235. The object information may be annotated on the obtained radiographic image by the annotation unit 206. The display control unit 207 may display the radiographic image annotated with the object information on the display unit 235.

The object-information determination unit 203 may determine object information including the information indicating the body part of the object in the optical image using the skeleton information of the object in the optical image, as described above. With the above configuration, the information processing apparatus 200 according to Related Example 2 can determine the body part of the object (the target of radiographic imaging) as the object information using the skeleton information estimated from the optical image. This enables the information processing apparatus 200 to more appropriately support the operator in determining whether the body part of the object is suitable using the determined object information. The determined object information may be used as additional information provided to the operator together with a live image in radiographic imaging for medical examinations, as in Related Example 1. Furthermore, the information processing apparatus 200 can appropriately estimate the skeleton information required in medical institutions or medical fronts without the need for collecting a large amount of training data, as in Related Example 1. In Related Example 2, the object information including information indicating the body part of the object is determined, information indicating the laterality and the body part of the object may be determined as object information.

### Related Example 3

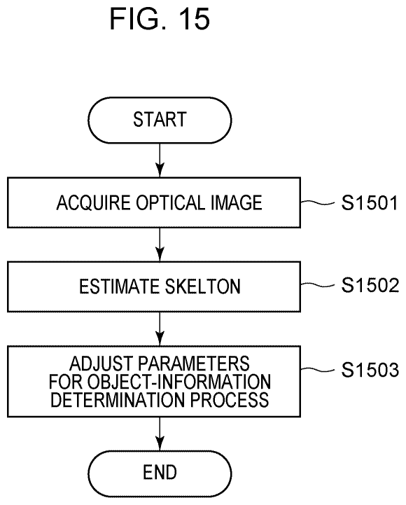

Referring to FIG. 15, a radiographic imaging system, an information processing apparatus, and a method for information processing according to Related Example 3 will be described. Related Example 3 describes a customizing (adjusting) process for recognizing the laterality and the body part of the target object from optical images acquired at a predetermined frame rate using a video camera as the camera 140 and outputting the laterality and the body part of the target object as object information.

Since the configuration of the radiographic imaging system and the information processing apparatus according to Related Example 3 is the same as the configuration of the radiographic imaging system and the information processing apparatus according to Related Examples 1 and 2, the description will be omitted using the same reference signs. The description of processing similar to that detailed in Related Examples 1 and 2 will be omitted.

### Processing Procedure

Referring to FIG. 15, processing steps of the adjusting process according to a rule-based algorithm for the object-information determination process according to Related Example 3 will be described. FIG. 15 is a flowchart showing the processing steps according to Related Example 3. When the adjusting process according to the rule-based algorithm for the object-information determination process according to Related Example 3 are started, the process proceeds to step S1501.

### Step S1501

In step S1501, the optical-image acquisition unit 201 controls the camera 140 to acquire an optical image of a radiographic imaging part including the object of radiographic imaging. In Related Example 3, the camera 140 is a video camera attached to the radiation generator. The camera 140 captures images of the object who takes an imaging posture on the radiation detector 130 placed on a supine table and outputs optical images at predetermined frame rate. Here, an example in which the radiation detector 130, the head 1002, the chest 1003, and the abdomen 1004 appear in the optical image 1000 will be described with reference to FIG. 10, as in Related Example 2.

### Step S1502

In step S1502, the skeleton estimation unit 202 estimates the skeleton information of the object by using the optical image acquired by the optical-image acquisition unit 201 as input data for the trained model. In Related Example 3, the skeleton estimation unit 202 performs whole-body skeleton estimation in which the left and right eyes, left and right shoulder joints, left and right hip joints, left and right elbows, left and right wrists, left and right knees, and left and right ankles are estimated as skeleton information, and the whole-body skeleton estimation is applied to the optical image 1000. As illustrated in FIG. 11, the skeleton estimation unit 202 can detect left and right eyes 1101 and 1102, left and right shoulder joints 1103 and 1104, and left and right hip joints 1105 and 1106 in the optical image 1000 as feature points, as in Related Example 2.

The trained model for use in Related Example 3 may also be a trained model obtained by additionally training a trained model for use in general-purpose skeleton estimation so as to output a feature point for each medical institution or medical front, similarly to the trained model according to Related Example 1. The training data may be prepared in advance, similarly to the training data according to Related Example 1.

### Step S1503

In step S1503, the object-information determination unit 203 adjusts the parameters of the rule-based algorithm for determining the body part of the target object of radiographic imaging in response to the input from the operator via the operation unit 234. More specifically, the display control unit 207 displays the skeleton information estimated by the skeleton estimation unit 202 on the display unit 235. The operator provides an instruction for the body part of the object to be output as object information to the information processing apparatus 200 via the operation unit 234 based on the skeleton information displayed on the display unit 235. The object-information determination unit 203 adjusts the various parameters of the rule-based algorithm, as shown in FIG. 12, in response to the instruction from the operator.

Examples of the parameters of the rule-based algorithm may include the threshold for the threshold process applied to the probability in step S1202 and the number of feature points serving as the reference for determination for branching off the process of step S1201. The parameters of the rule-based algorithm may include the radiation target field and the positional relationship between and the feature point to be kept and the radiation target field in step S1203, and the correspondence relationship between the remaining feature points and the body part to be output in step S1204. The positional relationship between the feature point to be kept and the radiation target field may include the order of closeness of the feature point to be kept to the radiation target field (for example, the closest or the second closest).

For example, assume that the operator inputs an instruction that the body part determined from the estimated skeleton information is “chest” via the operation unit 234 so to adjust the rule-based algorithm as shown in FIG. 12. In this case, the object-information determination unit 203 determines a radiation target field and identifies which of the left and right eyes 1101 and 1102, the left and right shoulder joints 1103 and 1104, and the left and right hip joints 1105 and 1106 is the feature point closest to the radiation target field. In the above example, either the shoulder joint 1103 or 1104 is the closest. For this reason, when the shoulder joint is closest to the radiation target field, the object-information determination unit 203 adjusts the parameters of the rule-based algorithm so as to determine the “chest” to be the object information and output it. In this case, for example, in step S1203, the object-information determination unit 203 may adjust the positional relationship between the feature point to be kept and the radiation target field so as to determine either the shoulder joint 1103 or 1104 to be the feature point to be kept. The object-information determination unit 203 may adjust the correspondence relationship between the remaining feature point and the name of the body part to be output in step S1204.

The object-information determination unit 203 may store, of the skeleton information output from the skeleton estimation unit 202, the coordinates of feature points whose probabilities are equal to or greater than the threshold in the storage 232 together with the body part information input by the operator, without calculating the radiation target field. In this case, for example, the coordinates of the feature point when the body part information is registered as “chest” or the coordinates of the feature point when the body part information is registered as “abdomen” are stored in the storage 232. In such a case, when determining the body part of the object by inputting a certain optical image, the object-information determination unit 203 may calculate the sum of the distances between the coordinates of feature points obtained by inputting the optical image and the coordinates of the stored feature points and determine a body part with the smallest sum to be object information.

A method for adjusting the parameters of the rule-based algorithm may be configured to prepare multiple combinations of parameters and select one of the combinations of parameters in response to an instruction from the operator. For example, the object-information determination unit 203 may select a combination of parameters with reference to a prepared lookup table to adjust the parameters. Examples of a combination of parameters may include a combination of parameters related to the process as shown in FIG. 12, a combination of parameters related to the process using the coordinates of feature points with a threshold or greater, and a combination of parameters related to the process as shown in FIG. 14. Another example of a combination of parameters is a combination of parameters related to the process of determining the laterality of the object as shown in FIGS. 3 and 6. The instruction from the operator may be an instruction for object information to be output or an instruction for a combination of parameters.

When the parameters of the rule-based algorithm are adjusted in step S1503, the series of processing steps end.

Related Example 3 describes an example in which the parameters of the rule-based algorithm for the process of determining the body part of object as the object information are adjusted. In contrast, the parameters of the rule-based algorithm may be adjusted as in Related Example 3 for the process of adjusting the laterality of the object as the object information according to Related Example 1. In this case, examples of the parameters of the rule-based algorithm may include the threshold in the threshold process applied to the probability in step S601 and the number of feature points serving as a criterion for the branching of the process in step S602. Other examples of the parameters of the rule-based algorithm may include the radiation target field and the positional relationship between the feature point to be kept and the radiation target field in step S603 and the correspondence relationship between the remaining feature point in step S604 and the laterality to be output. Even for the process for determining the body part and laterality of the object as object information, the parameters of the rule-based algorithm may be adjusted as in Related Example 3.

Thus, the object-information determination unit 203 according to Related Example 3 can adjust the parameters of the rule-based process based on the skeleton information of the object in the optical image and the object information obtained via the operation unit 234. Here, the parameters of the rule-based process may include at least one of the threshold for the threshold process for the selection of a feature point of the object in an optical image, the positional relationship between the feature point and the radiation exposure field, and the correspondence relationship between the feature point and at least one of the body part and the laterality of the object. This configuration enables the information processing apparatus 200 according to Related Example 3 to flexibly apply the object-information determination process to the operation and knowledge in medical institutions or medical fronts by adjusting the rules according to the operation and knowledge in the medical institutions or medical fronts.

Related Example 3 describes, as in Related Examples 1 and 2, the case where an optical image of a radiographic imaging part is acquired by the optical-image acquisition unit 201. In contrast, the optical image for use in adjusting the rule-based algorithm may be an optical image acquired in a virtual radiographic imaging part by imaging a human body phantom. In other words, the object-information determination unit 203 may adjust the parameters for rule-based processing based on skeleton information obtained using an optical image acquired by imaging a phantom and object information obtained via the operation unit 234. In this case, an image in which skeleton estimation in step S1502 is easier than when using an actual image can be acquired, allowing more appropriate adjustment of the parameters in step S1503.

The optical image used for the adjustment of the rule-based algorithm does not have to be an actually captured image. For example, a two-dimensional image may be used in which three-dimensional coordinates indicating a virtual skeleton position calculated from virtual object data using a three-dimensional modelling tool. In this case, the object-information determination unit 203 may use the skeleton information output from the skeleton estimation unit 202 based on the two-dimensional image to adjust the parameters. In other words, the object-information determination unit 203 can adjust the parameters of the rule-based process based on the skeleton information obtained by projecting the three-dimensional coordinates of virtual object data generated using a three-dimensional modelling tool onto two-dimensional image coordinates and the object information obtained via the operation unit 234. This method allows for relatively easy acquisition of an image in which the skeleton position of a desired posture is shown on the information processing apparatus 200, without the need to place the human body or the phantom in a predetermined posture. Such a two-dimensional image may also be used as input data for training data related to additional training of a trained model to be used by the skeleton estimation unit 202.

In Related Example 3, the object-information determination unit 203 adjusts the parameters of the rule-based algorithm for the object-information determination process in response to an instruction from the operator. In contrast, the information processing apparatus 200 may has a programming environment, and the operator may describe a program for the skeleton information output from the skeleton estimation unit 202 via the operation unit 234. Such a configuration may use a programming language, such as C language or Python. The information processing apparatus 200 may be configured to use a no-code development platform capable of building a rule-based algorithm using a graphical user interface. Furthermore, in recent years, it has become possible to generate programs by inputting instructions in natural language using an artificial intelligence (AI) chatbot, which is a type of generative AI. For this reason, the information processing apparatus 200 may be configured to utilize generative AI, such as AI chatbot.

The training data of a trained model is not limited to data obtained using the camera 140 used for actual imaging and may also include data obtained using an identical camera or a similar type of camera, depending on the desired configuration. The trained model for skeleton estimation according to Related Examples described above is regarded as extracting the magnitude of luminance values, the order and inclination of bright and dark areas, their positions, distributions, and continuity, etc. of the optical image as some features and using the features for estimating skeleton information.

The trained model for skeleton estimation may be provided in the information processing apparatus 200. Example of the inference engine (trained model) include software modules executed by a processor, such as a CPU, MPU, GPU, or field-programmable gate array (FPGA), and a circuit that performs a specific function, such as an ASIC. The inference engine may be provided in another device, such as a server, connected to the information processing apparatus 200. In this case, the information processing apparatus 200 can use the inference engine by connecting to the server or the like including the inference engine via any network such as the Internet. Examples of the server including the inference engine include a cloud server, a fog server, and an edge server. To configure a radio communication network within a facility, a site including the facility, or within a region including multiple facilities, radio waves in a dedicated wavelength band allocated exclusively to the facility, site, or region may be used to improve the reliability of the network. The network may be configured using wireless communication capable of high-speed communication, large-capacity communication, low-latency communication, or multiple simultaneous connections.

### First Embodiment

Related Examples 1 to 3 describe a method for performing skeleton estimation of the object on the assumption that only one object appears in the optical image. However, when multiple objects appear in the optical image, it is not possible to determine for which of the objects the skeleton estimation should be performed, and the process for the skeleton estimation may be stopped. This may make it impossible to determine whether the imaging posture of the object is suitable.

A first embodiment describes a method for appropriately determining whether the imaging posture of the object is suitable even when multiple objects appear in the optical image.

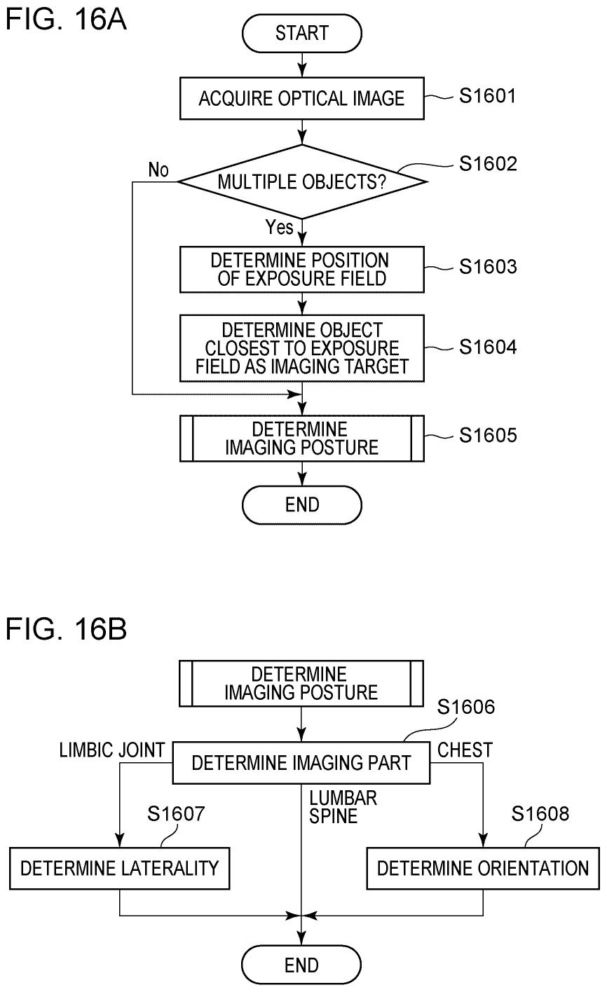

Referring to FIGS. 16A and 16B to FIG. 18, a radiographic imaging system, an information processing apparatus, and a method for information processing according to the first embodiment of the present disclosure will be described hereinbelow. This embodiment describes a process for determining an object closest to the radiation exposure field as the imaging target (the target of radiographic imaging) when multiple objects appear in an optical image acquired at a predetermined frame rate using the camera 140.

### Processing Procedure

A series of processing steps according to this embodiment will be described hereinbelow with reference to FIGS. 16A and 16B.

FIG. 16A is a flowchart showing the processing steps according to this embodiment. When the processing steps according to this embodiment are started, the process proceeds to step S1601.

### Step S1601

In step S1601, the optical-image acquisition unit 201 controls the camera 140 to acquire an optical image of a radiographic imaging part including the object of radiographic imaging. In this embodiment, the camera 140 is a video camera attached to the radiation generator. The camera 140 captures images of the object who takes an imaging posture on the radiation detector 130 placed in a standing position and outputs optical images at predetermined frame rate.

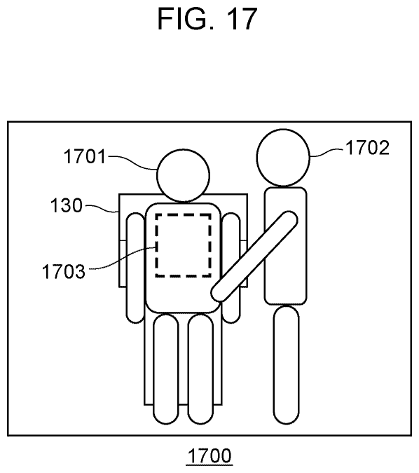

Here, an example in which the radiation detector 130, an object 1701, and an object 1702 appear in an optical image 1700 will be described with reference to FIG. 17. FIG. 17 illustrates an example of the optical images according to this embodiment.

### Step S1602

In step S1602, the human detection unit 208 and the image analysis unit 209 determine whether the number of objects to be the target of radiographic imaging is one or more than one by using the optical image acquired by the optical-image acquisition unit 201 as input data for the trained model.

If in step S1602 it is determined that the number of objects to be the target of radiographic imaging is more than one, the process proceed to step S1603.

In contrast, if in step S1602 it is determined that the number of objects to be the target of radiographic imaging is one, the process proceeds to step S1605.

In the example of FIG. 17, since the object-information determination unit 203 determines that the number of objects to be the target of radiographic imaging is two, the process proceeds to step S1603.

### Step S1603

In step S1603, the object-information determination unit 203 determines the position of a radiation target field (a radiation exposure field 1703) to be irradiated with radiation in the optical image 1700.

The position of the radiation exposure field 1703 in the optical image 1700 is identified based on information on the imaging distance included in the imaging order information. This is because, when the camera 140 is provided in the radiation generation unit 120, a change in the distance between the radiation generation unit 120 and the radiation detector 130 alters the radiation exposure field 1703 in the optical image 1700. One example of the information on the imaging distance is a source-to-image receptor distance (SID), which is the distance between the radiation generation unit 120 and the radiation detector 130. Another example is the intended use of the radiation detecting apparatus related to the SID (for use in standing position imaging, supine position imaging, or portable imaging). The intended use of the radiation detecting apparatus may also be expressed as position type.

The position of the radiation exposure field 1703 in the optical image 1700 may be calculated in consideration of the spatial positions of the radiation generation unit 120 and the camera 140, as in the first embodiment. The position of the radiation exposure field 1703 may be the position of the exposure field of the collimator lamp or the center of the optical image 1700. When the center position of image analysis has to be determined when the image analysis unit 209 performs an image analysis, the center position of the radiation exposure field may be used as the center position of the image analysis. The object-information determination unit 203 is an example of an identification unit that identifies the radiation exposure field.

In the above example, since the object 1701 is closer to the radiation exposure field 1703 than the object 1702, the object 1701 is determined to be the imaging target object, and the process proceeds to step S1604. Whether the object is close to the radiation exposure field 1703 is determined based on the distance between the center position of the radiation exposure field 1703 and the center of the object detection position (detection frame).

### Step S1604

In step S1604, the object-information determination unit 203 determines the object 1701, which is closest to the radiation exposure field 1703 in the optical image 1700, obtained in step S1603, to be the imaging target object.

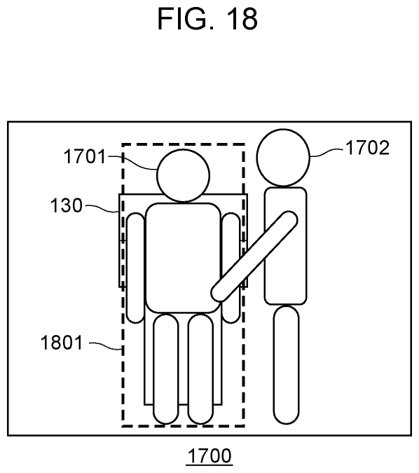

Here, the display control unit 207 may display the object 1701, which is determined to be the imaging target, with emphasis, for the purpose of showing the operator (the radiology technician or the like) the determination result in step S1604, as shown in FIG. 18. FIG. 18 illustrates an example of the emphasis in which an object detection frame 1801 enclosing the object 1701 is displayed.

### Step S1605

In step S1605, the consistency determination unit 204 determines whether the information on the imaging target object determined by the object-information determination unit 203 is consistent with the radiographic imaging order information. If they are not consistent, an alert such as “Check: Imaging part” or “Check: Field of view position” is displayed on the display unit 235. When they are consistent, a message such as “Consistent” is displayed on the display unit 235. Even when they are consistent, the message “consistent” does not have to be displayed on the display unit 235. In other words, a massage indicating inconsistent may be displayed on the display unit 235 only in an inconsistent status.

A specific procedure of step S1605 is shown in FIG. 16B. In step S1606, the consistency determination unit 204 determines whether the imaging target is consistent with the imaging part specified in the imaging order information. More specifically, the consistency determination unit 204 determines whether the imaging target at the center position of the image analysis is consistent with the imaging part specified in the imaging order information. If it is determined that they are consistent, the consistency determination unit 204 proceeds to step S1607 or S1608 or terminates the processing, depending on the imaging part.

For example, if the imaging part is a limbic joint, the process proceeds to S1607 and determines the laterality (left or right). In S1607, the consistency determination unit 204 determines whether the laterality of the imaging target is consistent with the laterality specified in the imaging order information. The laterality specified in the imaging order information is an example of information related to an imaging posture.

For example, if the imaging part is the chest, the process proceeds to S1608 to determine the orientation (for example, PA or AP). In S1608, the consistency determination unit 204 determines whether the orientation of the imaging target is consistent with the orientation specified in the imaging order information. The orientation specified in the imaging order information is an example of the information regarding the imaging posture.

For example, if the imaging part is the lumbar spine, the determination of the laterality and orientation is not performed, and the processing is terminated.

When the process of step S1605 is completed, the series of processes in this embodiment ends.

Thus, even when multiple objects appear in the optical image, the object-information determination unit 203 can determine the imaging target.

The above configuration allows the information processing apparatus 200 according to this embodiment to determine the imaging target even when multiple objects appear in the optical image.

In other words, even when multiple objects appear in the optical image, the information processing apparatus 200 according to this embodiment can appropriately determine whether the imaging posture of the object is suitable.

Although this embodiment describes a method for determining the imaging target based on the position of the radiation exposure field, the method for determining the position of the radiation exposure field is not limited to the above. For example, the position of the radiation exposure field may be determined by the image recognition of the radiation detector 130 that appears in the optical image. The image recognition of the radiation detector 130 may be performed using a trained neural network model. When multiple radiation detectors are recognized, a radiation detector closest to the center of the optical image may be selected.

This embodiment may additionally include the step of analyzing the body motion of the object. The body motion refers to a sudden motion that is generated by the sneezing of the object. For example, after in S1605 it is determined that the posture of the object is consistent with the imaging order information, the process proceeds to the step of analyzing the body motion of the object. If the body motion is detected in the step, the posture of the object may be inconsistent with the imaging order information, and therefore, an alert prompting the user to check the object's posture. If the body motion is detected, for example, the steps S1601 to S1605 are executed again. Although this embodiment describes the case where the imaging target is a human body, this disclosure is also applicable to a case where the imaging target is not a human body. For example, this disclosure is also applicable to a case where the imaging target is a baggage in a radiographic baggage inspection. Alternatively, the disclosure is applicable to a case where the imaging target is a product in a radiographic product inspection (for example, inspection of electrical boards or pipes). When the imaging target is a baggage, the analysis unit analyzes, for example, whether there are hazardous materials in the baggage. When the imaging target is a product, the analysis unit analyzes, for example, whether the electrical board has a defect or whether the welded part of the pipes has a defect.

### Second Embodiment

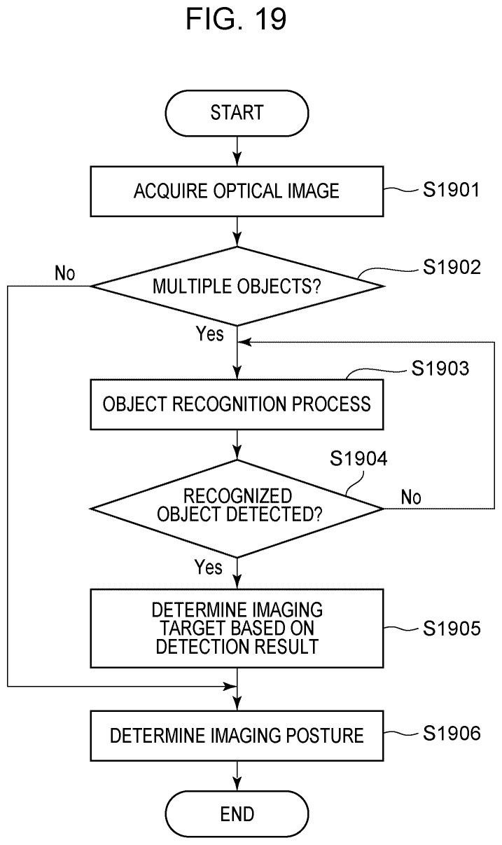

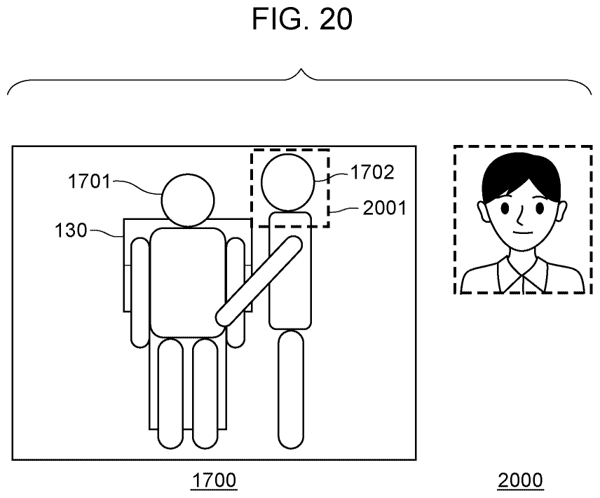

Referring to FIGS. 19 and 20, a radiographic imaging system, an information processing apparatus, and a method for information processing according to a second embodiment of the present disclosure will be described hereinbelow.

This embodiment describes a process for determining the imaging target by recognizing the features of the object when multiple objects appear in optical images acquired at a predetermined frame rate using the camera 140.

### Processing Procedure

A series of processing steps according to this embodiment will be described hereinbelow with reference to FIG. 19. FIG. 19 is a flowchart showing the processing steps according to this embodiment. When the processing steps according to this embodiment are started, the process proceeds to step S1901.

### Step S1901

In step S1901, the optical-image acquisition unit 201 controls the camera 140 to acquire an optical image of a radiographic imaging part including the object of radiographic imaging. In this embodiment, the camera 140 is a video camera attached to the radiation generator. The camera 140 captures images of the object who takes an imaging posture on the radiation detector 130 placed in a standing position and outputs optical images at predetermined frame rate.

Here, an example in which the radiation detector 130, an object 1701, and an object 1702 appear in an optical image 1700 will be described with reference to FIG. 20. FIG. 20 illustrates an example of the optical images according to this embodiment.

### Step S1902

In step S1902, the human detection unit 208 and the image analysis unit 209 determine whether the number of objects to be the target of radiographic imaging is one or more than one by using the optical image acquired by the optical-image acquisition unit 201 as input data for the trained model.

If in step S1902 it is determined that the number of objects to be the target of radiographic imaging is more than one, the process proceed to step S1903.

In contrast, if in step S1902 it is determined that the number of objects to be the target of radiographic imaging is one, the process proceeds to step S1906.

In the example of FIG. 20, since the object-information determination unit 203 determines that the number of objects to be the target of radiographic imaging is two, the process proceeds to step S1903. The objects 1701 and 1702 in FIG. 20 are examples of a first human body and a second human body, respectively.

### Step S1903

In step S1903, the object-information determination unit 203 performs a process for recognizing the features of the individual objects in the optical image 1700.

In the process of recognizing the features of the individual objects in the optical image 1700, the features are recognized based on the information for recognizing the features, which is stored in the storage 232.

Examples of the information for recognizing the features may include the face image of the patient (object), the face image of the radiology technician (photographer), information on a specific marker (for example, a wristband) assigned to the patient, information on the characteristics of inspection clothing (color, shape, etc.), and information on the characteristics of the radiation protection suit (color, shape, etc.).

In this embodiment, a face image 2000 of the radiology technician is stored in the storage 232 as the information for recognizing the features, and the recognition process is performed using the face image 2000.

### Step S1904

In step S1904, the object-information determination unit 203 determines whether the object was recognized in the recognition process of step S1903.

If in step S1904 it is determined that the object was recognized, the process proceeds to step S1905.

In contrast, if in step S1904 it is determined that no object was recognized, the process returns to step S1903, where the recognition process is performed again.

In this embodiment, since the object-information determination unit 203 determines that the face image 2000 of the radiology technician, which is stored in the storage 232, was recognized as the object 1702, the process proceeds to step S1905.

Here, the display control unit 207 may display a recognized-object frame 2001 that encloses the face region of the recognized object 1702, as shown in FIG. 20, to show the result of step S1904 to the operator.

### Step S1905

In step S1905, the object-information determination unit 203 determines the imaging target object in the optical image 1700 based on the recognition result of step S1904.

In this embodiment, since it is determined that the object 1702 is a radiology technician from the result of step S1904, the object 1701 is determined to be the imaging target object. When in step S1903 a recognition process is performed using the face image of the object 1701 (the face image of the patient), and if in step S1904 the object 1701 is recognized, the object-information determination unit 203 determines the object 1701 to be the imaging target object.

### Step S1906

In step S1906, the consistency determination unit 204 determines whether the information on the imaging target object determined by the object-information determination unit 203 is consistent with the radiographic imaging order information.

When the process of step S1906 is completed, the series of processes according to this embodiment ends.

Thus, the object-information determination unit 203 can determine the imaging target even when multiple objects appear in the optical image.

The above configuration allows the information processing apparatus 200 according to this embodiment to appropriately determine whether the imaging posture of the object is suitable even when multiple objects appear in the optical image.

### Third Embodiment

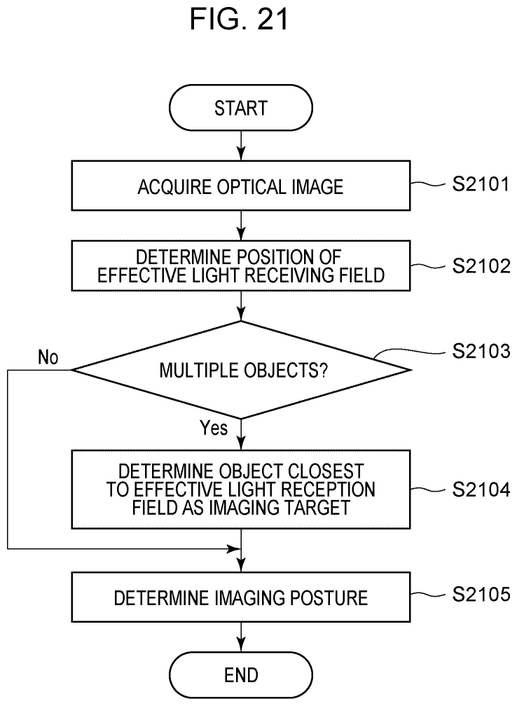

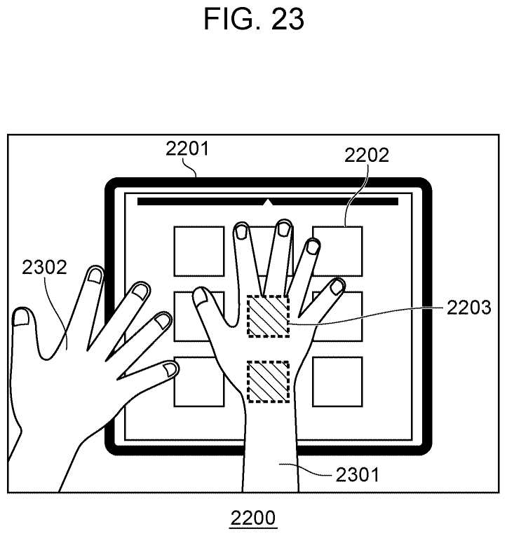

Referring to FIGS. 21 and 23, a radiographic imaging system, an information processing apparatus, and a method for information processing according to a third embodiment of the present disclosure will be described hereinbelow.

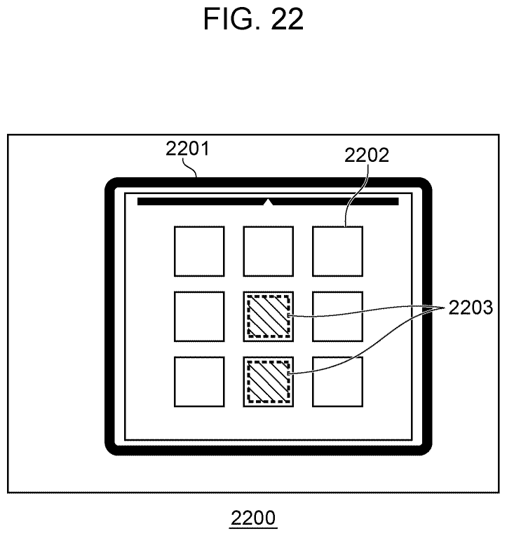

A radiation detector 130 of this embodiment has an automatic exposure control (AEC) function. The radiation detector 130 of this embodiment has multiple radiation detection regions (light reception fields) on the detection surface. In imaging using the radiation detector 130 of this embodiment, the following description explains a process for determining the imaging target based on the positions of the light reception fields when multiple objects are captured in the optical image acquired at a predetermined frame rate by the camera 140. In the radiographic imaging using automatic exposure control, a light reception field to be used is selected from among multiple light reception fields. FIG. 22 illustrates an example in which two light reception fields 2203 are selected from nine light reception fields. The selected light reception fields 2203 may be regarded as imaging target regions. In other words, the selected light reception fields 2203 may be regarded as radiation exposure fields.

### Processing Procedure

A series of processing steps according to this embodiment will be described with reference to FIG. 21.

FIG. 21 is a flowchart showing the processing steps according to this embodiment. When the processing steps according to this embodiment are started, the process proceeds to step S2101.

### Step S2101

In step S2101, the optical-image acquisition unit 201 controls the camera 140 to acquire an optical image of a radiographic imaging part including the object of radiographic imaging. In this embodiment, the camera 140 is a video camera attached to the radiation generator. The camera 140 captures images of a radiation detector 2201 placed in a horizontal position and outputs optical images at predetermined frame rate.

Here, an example in which the radiation detector 2201 and light reception fields 2202 appear in an optical image 2200 will be described with reference to FIG. 22. FIG. 22 illustrates an example of the optical images according to this embodiment.

### Step S2102

In step S2102, the object-information determination unit 203 determines the position of the light reception field to be used in automatic exposure control from the optical image acquired by the optical-image acquisition unit 201 based on the information on the light reception field for use in automatic exposure control of the radiation detector 2201, the information being specified in the imaging order information.

The display control unit 207 may emphatically display the position of the light reception field for use in the automatic exposure control acquired in step S2102 of FIG. 22. FIG. 22 illustrates an example in which the light reception fields 2203 for use with hatching.

### Step S2103

In step S2103, the human detection unit 208 and the image analysis unit 209 determine whether there is a single object or multiple objects that can be the target of radiographic imaging by using the optical image acquired by the optical-image acquisition unit 201 as input data for the trained model.

Here, a case is described with reference to FIG. 23 in which the radiation detector 2201, the light reception fields 2202, an object 2301, and an object 2302 appear in the optical image 2200. FIG. 23 illustrates an example of the optical images according to this embodiment.

If in step S2103 it is determined that the number of objects to be the target of radiographic imaging is more than one, the process proceeds to step S2104.

In contrast, if in step S2103 it is determined that the number of objects to be the target of radiographic imaging is one, the process proceeds to step S2105.

In this embodiment, since the object-information determination unit 203 determines that the two objects 2301 and 2302 can be the target of radiographic imaging, as shown in FIG. 23, the process proceeds to step S2104.

### Step S2104

The object-information determination unit 203 determines that the object closest to the positions of the light reception fields 2203 for use in automatic exposure control, determined in step S2102, is the imaging target object.

In this embodiment, the object-information determination unit 203 determines that the object 2301 closest to the light reception fields 2203 for use in automatic exposure control is the imaging target, as illustrated in FIG. 23.

### Step S2105

In step S2105, the consistency determination unit 204 determines whether the information on the imaging target object determined by the object-information determination unit 203 is consistent with the radiographic imaging order information.

When the process of step S2105 is completed, the series of processes ends.

Thus, the object-information determination unit 203 can determine the imaging target even when multiple objects appear in the optical image.

The above configuration allows the information processing apparatus 200 according to this embodiment to appropriately determine whether the imaging posture of the object is suitable even when multiple objects appear in the optical image.

### Fourth Embodiment

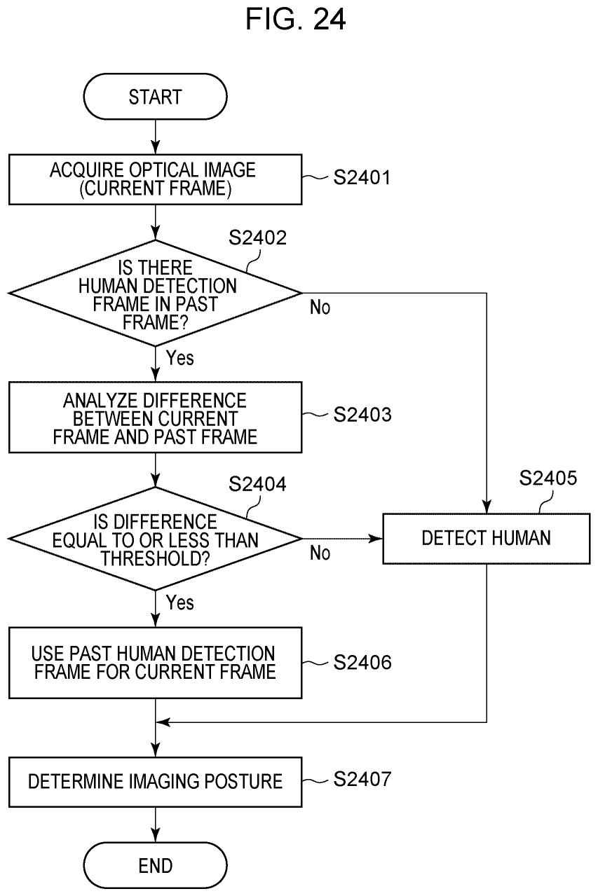

Referring to FIG. 24, a radiographic imaging system, an information processing apparatus, and a method for information processing according to a fourth embodiment of the present disclosure will be described hereinbelow.

In this embodiment, the information processing apparatus 200 acquires optical images at a predetermined frame rate using the camera 140. Next, the information processing apparatus 200 detects an object from the optical images.

The information processing apparatus 200 detects the imaging target by comparing the current frame with a past frame before the current frame. Referring to FIG. 24, a specific processing procedure of the information processing apparatus 200 will be described. The current frame is an example of a first optical image. The past frame is an example of a second optical image.

### Step S2401

In step S2401, the optical-image acquisition unit 201 controls the camera 140 to acquire an optical image of a radiographic imaging part including the object of radiographic imaging. The acquired optical image is referred to as a current frame in the following description. In this embodiment, the camera 140 is a video camera attached to the radiation generator and outputs optical images at a predetermined frame rate.

### Step S2402

In step S2402, the image analysis unit 209 checks the past frame before the current frame, the past frame being stored in the storage 232. Specifically, the image analysis unit 209 acquires object information in the past frame and determine whether an object is detected. If an object is detected, the process proceeds to S2403. If in S2402 no object is detected, the process proceeds to S2405.

### Step S2403

In step S2403, the image analysis unit 209 acquires the detection position (detection frame) of the object in the past frame images stored in the storage 232. Next, the image analysis unit 209 analyzes the difference between the current frame image and the past frame images to determine whether the object moved between the frames.

Specifically, if a detection position (detection frame) in which the object is detected is present in the past frame, the image analysis unit 209 sets the detection position (detection frame) as the detection position (detection frame) in the current frame. Next, the image analysis unit 209 determines the change by comparing the average luminance and variance of the region of the detection frame set in the current frame with the average luminance and variance of the detection frame of the past frame. The detection position (detection frame) in the current frame is an example of a first region. The detection position (detection frame) of the past frame is an example of a second region.

For the analysis of the difference, edge detection or optical flow analysis may be used. The edge detection can enhance the edges using methods such as the Sobel filter or the Canny method, thereby clarifying the boundary of change. The boundary of motion is not limited to the boundary of the motion of the object. For example, in the configuration in which the camera 140 is attached to the radiation generation unit 120, when the radiation generation unit 120 is moved by the radiology technician, not only the object but also the background and the standing position stand move. In other words, the motion includes the motion of the background and the standing position stand. In optical flow analysis, determining the motion of any point in the image in vectors allows providing more detailed motion information.

Here, a method of comparison using the average luminance and variance will be described. The image analysis unit 209 compares the luminance value of the object in the detection frame of the current frame for each pixel with the luminance value of the object in the detection frame of the past frame for each pixel to calculate the difference. In this case, the image analysis unit 209 calculates the total sum or the average of the differences between the luminance values of the pixels in the detection frames, thereby evaluating whether there is a motion between the detection frames. The image analysis unit 209 also calculates the variance in the luminance values of the pixels in the detection frames and compares the variations to determine whether there are local variations in luminance between the detection frames. This enables the detection of motion while suppressing the effects of minute motion and noise.

The image analysis unit 209 may calculate the average luminance for each of divided blocks (divided regions) of the region of the object detection frame, rather than comparing (calculating the difference between) the pixels. In the method of dividing into blocks (block division), the image analysis unit 209 compares the average luminance of the blocks of the current frame with the average luminance of the blocks of the past frame. Next, as a result of the difference calculation, the image analysis unit 209 determines the proportion of blocks with a difference exceeding a predetermined threshold, thereby evaluating the presence of motion.

This method enables the detection of partial motion or change in a specific region. The block-based analysis has also the advantage of improved robustness against noise and minute changes between frames, as compared with the method of calculating the difference pixel by pixel. For example, the block-based analysis can detect luminance changes within a block in which only part of the object moves and therefore can improve the accuracy of motion detection. The block-based analysis detects variations in luminance in each block by evaluating the variance in luminance (luminance variance) in each block, thereby allowing detailed identification of the characteristics of the motion. For example, when the luminance variance is large, it can be determined that motion is likely to occur in the block. In contrast, when the luminance variance is small, it can be determined that motion is unlikely to occur in the block. This method is effective, for example, to detect minute motion of the object.

The image analysis unit 209 may detect a motion using not only a temporal change but also spatial frequency information. Specifically, using phase-only correlation (POC) in a frequency space allows the motion between frames to be detected with high accuracy. The POC is a method for detecting the magnitude and direction of motion by extracting frame-to-frame phase information using Fourier transformation and calculating the correlation. The method using the POC can effectively detect motion that is difficult to detect through conventional difference comparison in a spatial region, such as a small motion and a partially directional motion. Since the method using the POC can also detect minute changes in object position with high accuracy, it may also be effective in detecting the minute motion and the relative motion of the object relative to the background.

Thus, a combination of multiple methods may be used to determine the presence of motion between frames. The past frame may be the previously analyzed frame (the frame one step before the current frame). However, the frame two steps or more before the current frame may be used in the period during which no motion is detected in the previously analyzed frame. In other words, the past frame is not necessarily be limited to the previous frame. This allows for the evaluation of the presence of motion more broadly (temporally broadly).

An example has been described in which the difference between the object detection frame of the current frame and the object detection frame of the past frame is calculated, but it is also possible to calculate the difference between the entire frames. The difference may be calculated for a predetermined field (for example, a radiation exposure field identified based on the imaging order). The radiation exposure field is identified based on the information on the imaging distance included in the imaging order information, as described in step S1603. The image analysis unit 209 may calculate the difference between the radiation exposure field of the past frame and the radiation exposure field of the current frame corresponding to the radiation exposure field of the past frame. This enables the analysis of overall motion including motion other than that of the object (for example, motion indicating changes in the background).

### Step S2404

If the difference between the frames analyzed in step S2403 is less than or equal to a predetermined threshold, the process proceeds to step S2406. In contrast, if the difference between the frames is greater than the threshold, the process proceeds to step S2405.

In step S2402, the image analysis unit 209 may determine the reliability of the object detection frame, rather than object information in the past frame. If the reliability is low, the process shifts from step S2402 to step S2405.

### Step S2405

In step S2405, the human detection unit 208 detects the object from the current frame. The human detection is the same as in the other embodiments described above, and a detailed description will be omitted.

### Step S2406

In step S2406, the image analysis unit 209 uses the object detection position (detection frame) in the past frame stored in the storage 232 as the detection frame of the current frame. In other words, the image analysis unit 209 outputs the detection frame (analysis result) of the past frame as the detection frame (analysis result) of the current frame. This allows for reduction in the processing load for human detection for each frame. Depending on the lighting environment of the imaging room, the contrast between the background and the object may be decreased. In conventional methods, the decrease in the contrast between the background and the object causes unstable recognition of the object itself, resulting in unstable display of the human detection frame. The unstable display refers to, for example, a case where the human detection frame is intermittently shown or not shown and a case where the position of the human detection frame changes in a short time. In the method of the present disclosure, when the difference between the current frame and the past frame is small, the detection frame of the past frame is used, thereby stabilizing the display of the human detection frame.

### Step S2407

In step S2407, the consistency determination unit 204 determines the imaging posture. Since the operation in step S2407 is the same as in the other embodiments, a detailed description will be omitted. If in S2404 it is determined that there is no motion (the difference between the current frame and the past frame is small), the determination result in the past frame image stored in the storage 232 may be used also for the determination of the imaging posture.

Although the present disclosure has been described with reference to the embodiments and examples, the disclosure is not limited to these embodiments and examples. The scope of the present disclosure also encompasses modifications that do not depart from the gist of the present disclosure, as well as equivalents thereof. The above-described embodiments and examples may be appropriately combined without departing from the spirit of the present disclosure.

According to an embodiment of the present disclosure, even when multiple objects appear in the optical image, it can appropriately be determined whether the imaging posture of the object is suitable.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present disclosure has been described with reference to exemplary embodiments, it is to be understood that the disclosure is not limited to the disclosed exemplary embodiments. The scope of the following claims is to be accorded the broadest interpretation so as to encompass all such modifications and equivalent structures and functions.

This application claims the benefit of Japanese Patent Application No. 2024-100393, filed Jun. 21, 2024, and No. 2024-206046 filed Nov. 27, 2024, which are hereby incorporated by reference herein in their entirety.

What is claimed is:

1. An information processing apparatus comprising:
at least one memory storing instructions; and
at least one processor that, upon execution of the instructions, is configured to operate as:
an acquisition unit configured to acquire imaging order information and an optical image related to radiographic imaging;
an identification unit configured to identify a radiation exposure field using the imaging order information and the optical image;
a detection unit configured to detect a human body part in the optical image; and
an analysis unit configured to, when a plurality of human body parts is detected in the optical image, analyze a human body part closest to the radiation exposure field using the optical image.

2. The information processing apparatus according to claim 1,
wherein the imaging order information includes information on an imaging distance, and
wherein the identification unit identifies the radiation exposure field using the information on the imaging distance.

3. The information processing apparatus according to claim 1, wherein the information on the imaging distance comprises an intended use of a radiation detecting apparatus.

4. The information processing apparatus according to claim 1,
wherein the imaging order information includes information on an imaging part, and
wherein the analysis unit analyzes whether the human body part closest to the radiation exposure field is consistent with the information on the imaging part.

5. The information processing apparatus according to claim 1,
wherein the imaging order information includes information on an imaging posture, and
wherein the analysis unit analyzes whether a posture of the human body part closest to the radiation exposure field is consistent with the information on the imaging posture.

6. The information processing apparatus according to claim 5,
wherein the imaging order information includes information on a light reception field for use in automatic exposure control, and
wherein the analysis unit analyzes whether a posture of a human body part closest to the light reception field is consistent with the information on the imaging posture.

7. The information processing apparatus according to claim 1, further comprising a display control unit configured to, when a plurality of human body parts is detected in the optical image, display, on a display device, a human body part closest to the radiation exposure field with emphasis.

8. A radiographic imaging system comprising:
a radiation detecting apparatus configured to detect radiation; and
the information processing apparatus according to claim 1, the information processing apparatus being communicably connected to the radiation detecting apparatus.

9. A method for information processing, comprising:
acquiring imaging order information and an optical image related to radiographic imaging;
identifying a radiation exposure field using the imaging order information and the optical image;
detecting a human body part in the optical image; and
when a plurality of human body parts is detected in the optical image,
analyzing a human body part closest to the radiation exposure field using the optical image.

10. An information processing apparatus comprising:
at least one memory storing instructions; and
at least one processor that, upon execution of the instructions, is configured to operate as:
an acquisition unit configured to acquire an optical image related to radiographic imaging;
a detection unit configured to detect a region including a human body part in the optical image; and
an analysis unit configured to output an analysis result of the region detected by the detection unit,
wherein, when a difference between a first optical image acquired by the acquisition unit and a second optical image acquired before the first optical image is larger than a predetermined threshold, the analysis unit outputs an analysis result of a first region detected in the first optical image as an analysis result of the first optical image, and when the difference is smaller than a predetermined threshold, the analysis unit outputs an analysis result of a second region detected in the second optical image as an analysis result of the first optical image.

11. The information processing apparatus according to claim 10, wherein the difference is a difference between the second region in the second optical image and a region corresponding to the second region in the first optical image.

12. The information processing apparatus according to claim 10, wherein the difference is a difference between an average luminance or variance of the second region in the second optical image and an average luminance or variance of a region corresponding to the second region in the first optical image.

13. The information processing apparatus according to claim 10, wherein the difference is a difference between an average luminance or variance of each divided region of the second region in the second optical image and an average luminance or variance of a divided region corresponding to the divided region in the first optical image.

14. The information processing apparatus according to claim 10, wherein the difference is a difference between a second exposure field in the second optical image, the second exposure field being identified based on imaging order information, and a field corresponding to the second exposure field in the first optical image.

15. A non-transitory computer readable storage medium storing a program for causing a computer to execute the method for information processing according to claim 9.
